SEQUENCE LISTING (31 sequences)

SEQ ID NO: 1 - DNA, 17 bp, Human
ctgtttccgcgtgccct

SEQ ID NO: 2 - DNA, 17 bp, Human
gccacagcccaggcctt

SEQ ID NO: 3 - DNA, 19 bp, Human
gagacatcggactgcagat

SEQ ID NO: 4 - DNA, 21 bp, Human
gtggtggcaatgtccacgtta

SEQ ID NO: 5 - DNA, 21 bp, Human
ctggactggcaatagcaagct

SEQ ID NO: 6 - DNA, 20 bp, Human
agagggtcaatggcgttctg

SEQ ID NO: 7 - DNA, 23 bp, Human
tggaacgtgtgaaagctgagtct

SEQ ID NO: 8 - DNA, 23 bp, Human
catctgctcattctttctttgca

SEQ ID NO: 9 - DNA, 19 bp, Human
cacttggtcctgcgcttga

SEQ ID NO: 10 - DNA, 24 bp, Human
caattgggaatgcaacaactttat

SEQ ID NO: 11 - DNA, 20 bp, Human
gacgggcaaagatgtcagga

SEQ ID NO: 12 - DNA, 20 bp, Human
gcccgtttggccttttctac

SEQ ID NO: 13 - DNA, 21 bp, Human
tcttaggtgtgccaaaatcgg

SEQ ID NO: 14 - DNA, 21 bp, Human
tgtcagggtggtacttcatgg

SEQ ID NO: 15 - DNA, 20 bp, Human
tgctgagtccgcagcaggtg

SEQ ID NO: 16 - DNA, 20 bp, Human
gctggcaggctctggggaag

SEQ ID NO: 17 - DNA, 21 bp, Human
ccctccagaacatctccccat

SEQ ID NO: 18 - DNA, 21 bp, Human
acatgactgggtccaagttgt

SEQ ID NO: 19 - DNA, 21 bp, Human
cacgacaggcttgctgaatga

SEQ ID NO: 20 - DNA, 21 bp, Human
cttccgtaaacacgtccagtg

SEQ ID NO: 21 - DNA, 21 bp, Human
acaggagcttggtcagctaag

SEQ ID NO: 22 - DNA, 20 bp, Human
catgcaccgtctcatttgcg

SEQ ID NO: 23 - DNA, 20 bp, Human
gaggacgcggtgtcagaaat

SEQ ID NO: 24 - DNA, 22 bp, Human
ggttgctctgctatgagtctct

SEQ ID NO: 25 - DNA, 20 bp, Human
tacccccaggagaagattcc

SEQ ID NO: 26 - DNA, 20 bp, Human
gccatctttggaaggttcag

SEQ ID NO: 27 - DNA, 19 bp, Human
cccaggaggagtttggcaa

SEQ ID NO: 28 - DNA, 21 bp, Human
tgctggatcatctcatggagg

SEQ ID NO: 29 - DNA, 22 bp, Human
aagcagttagcaaggaaaggtc

SEQ ID NO: 30 - DNA, 26 bp, Human
gacatatactccatgtagggaagtga

SEQ ID NO: 31 - DNA, 23 bp, Human
cggtgcgtagtctggagctacgg

(19) United States
(12) Patent Application Publication
(10) Pub. No.: US 2024/0033277 A1
(43) Pub. Date: Feb. 1, 2024

(54) METHODS FOR TREATING AUTOIMMUNE DISEASES

(71) Applicant: NEW YORK SOCIETY FOR THE RELIEF OF THE RUPTURED AND CRIPPLED, MAINTAINING THE HOSPITAL FOR SPECIAL, New York, NY (US)

(72) Inventors: Vidyanath Chaudhary, New York, NY (US); Franck Barrat, Eastchester, NY (US)

(21) Appl. No.: 18/265,194

(22) PCT Filed: Dec. 2, 2021
(86) PCT No.: PCT/US2021/061619

§ 371 Date: Jun. 2, 2023

Related U.S. Application Data
(60) Provisional application No. 63/121,133, filed on Dec. 3, 2020.

(51) Int. Cl. A61K 31/7072; A61K 31/343; A61K 31/415; A61K 31/405; A61K 31/501; A61K 31/19; A61K 45/06; A61P 37/06

(52) U.S. Cl. CPC A61K 31/7072; A61K 31/343; A61K 31/415; A61K 31/405; A61K 31/501; A61K 31/19; A61K 45/06; A61P 37/06

(57) ABSTRACT

This disclosure features methods to treat autoimmune diseases using compounds that activate the unfolded protein response (UPR) and/or compounds that disrupt the tricarboxylic acid (TCA) cycle in immune cells such as plasmacytoid dendritic cells. The disclosure also features method of reducing production of inflammatory cytokines or chemokines by immune cells.

### CROSS-REFERENCE TO RELATED APPLICATION

This application claims the benefit of priority of U.S. Provisional Appl. No. 63/121,133, filed Dec. 3, 2020, the content of which is incorporated herein by reference in its entirety.

### SEQUENCE LISTING

The present specification is being filed with a computer readable form (CRF) copy of the Sequence Listing. The CRF entitled SequenceListing.txt, which was created on Nov. 18, 2021 and is 4.58 bytes in size, is incorporated herein by reference in its entirety.

### TECHNICAL FIELD

This disclosure relates to compositions and methods for the treatment of autoimmune diseases (e.g., systemic sclerosis and systemic lupus erythematosus).

### BACKGROUND

Up to 24 million Americans (more than 7% percent of the US population) suffer from an autoimmune disease—and the prevalence is rising (Progress in Autoimmune Disease Research, NIH, 2005). In multiple autoimmune diseases, plasmacytoid dendritic cells (pDCs) are chronically activated and can secrete extraordinary levels of type I IFN (IFN-I) when sensing nucleic acids from pathogens or from self (1-4). This response is central to the ability of pDCs to contribute to the control of viral infections (5, 6), but it can also contribute significantly to autoimmune diseases (3, 4).

Most autoimmune diseases have no standard medical treatment and have very few approved drugs for medical uses. Immunosuppressive drugs, which are presently considered as the golden standard for treating autoimmune disorder patients, are mostly associated with harmful side-effects, and long-term use of these medicines can potentially increase the risk of developing deadly infections and cancers.

Given that current treatments have tremendous shortcomings, there is a great need to understand the mechanisms of autoimmune diseases and develop efficacious therapies to treat such diseases.

### SUMMARY

This disclosure relates to methods for treating autoimmune conditions in a human subject using a compound that activates the Unfolded Protein response (UPR) and/or a compound that disrupts the tri-carboxylic acid (TCA) cycle in cells (e.g., dendritic cells, macrophages, T cells, B cells). The disclosure also features methods of reducing production of inflammatory cytokines or chemokines by cells (e.g., dendritic cells) in a human subject.

In a first aspect, the disclosure features a method of treating an autoimmune disease in a human subject in need thereof, the method comprising administering to the subject a therapeutically effective amount of a compound that activates the Unfolded Protein response (UPR) in immune cells in the subject. In one embodiment, the disclosure features the use of a compound that activates the Unfolded Protein response (UPR) in immune cells in the subject to treat an autoimmune disease in the subject.

In a second aspect, the disclosure features a method of treating an autoimmune disease in a human subject in need thereof, the method comprising administering to the subject a therapeutically effective amount of a compound that disrupts the tri-carboxylic acid (TCA) cycle in immune cells. In one embodiment, the disclosure features the use of a therapeutically effective amount of a compound that disrupts the tri-carboxylic acid (TCA) cycle in immune cells in the subject to treat an autoimmune disease in the subject.

In a third aspect, the disclosure features a method of reducing production of inflammatory cytokines or chemokines by immune cells in a human subject in need thereof, the method comprising administering to the subject, or contacting the immune cells in the subject with a therapeutically effective amount of a compound that activates the Unfolded Protein response (UPR) in immune cells in the subject.

In a fourth aspect, the disclosure features a method of reducing production of inflammatory cytokines or chemokines by immune cells in a human subject in need thereof, the method comprising administering to the subject, or contacting the immune cells in the subject with a therapeutically effective amount of a compound that disrupts the tri-carboxylic acid (TCA) cycle in immune cells in the subject.

In some embodiments, the immune cells are dendritic cells, macrophages, T cells, B cells, natural killer cells, and/or neutrophils. In some embodiments, the dendritic cells are plasmocytoid dendritic cells. In some embodiments, the dendritic cells express one or more of CD123, CD303 (BDCA2), CD304 (BDCA4), and immunoglobulin-like transcript 7 (ILT7). In some embodiments, the dendritic cells do not express the lineage-associated markers (Lin) CD3, CD19, CD14, CD16 and CD11c.

In some embodiments, the compound activates the IRE1α-XBP1 signaling branch of the UPR in immune cells. In some embodiments, the compound that activates the UPR is tunicamycin, thapsigargin, or IXA4.

In some embodiments, the compound that disrupts the tri-carboxylic acid (TCA) cycle is (a) a compound of Formula I

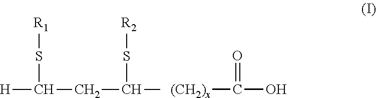

or a pharmaceutically acceptable salt thereof, wherein R₁ and R₂ are independently selected from the group consisting of acyl defined as R₃C(0)-, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10,
(b) UK5099 (PF-1005023)

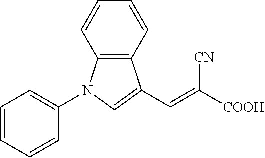 [SMILES: N#C/C(=C\c1cn(-c2ccccc2)c2ccccc12)C(=O)O]

or (c) CB839 (Telagenastat)

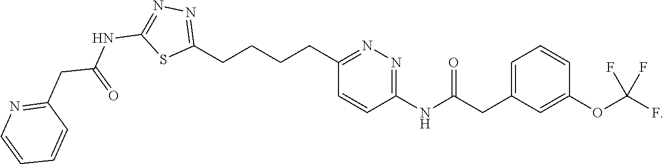 [SMILES: O=C(Cc1cccc(OC(F)(F)F)c1)Nc1ccc(CCCCc2nnc(NC(=O)Cc3ccccn3)s2)nn1]

In some embodiments, R₁ and R₂ are benzyl or benzoyl.

In some embodiments, the compound of Formula I is

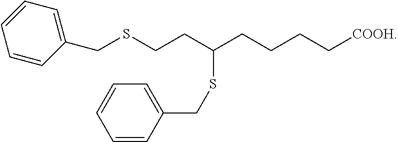 [SMILES: O=C(O)CCCCC(CCSCc1ccccc1)SCc1ccccc1]

In some embodiments, the compound of formula I is 6,8-bis-benzylthio-octanoic acid.

In some embodiments, the autoimmune disease is systemic sclerosis (scleroderma), systemic lupus erythematosus (SLE), rheumatoid arthritis (RA), Sjogren's syndrome, discoid lupus, cutaneous lupus, lupus nephritis, inflammatory bowel disease, psoriasis, type I diabetes, dermatomyositis, or polymyositis.

In some embodiments of any of the above aspects, the subject is concurrently treated with one or more agents selected from the group consisting of a nonsteroidal anti-inflammatory drug (NSAID), an immunosuppressant, a corticosteroid, an antimalarial, a fusion protein, and an antibody.

In some embodiments, the immunosuppressant is methotrexate, mycophenolate mofetil (MMF), cyclophosphamide, cyclosporin, or azathioprine. In some embodiments, the antimalarial is hydroxychloroquine or chloroquine. In some embodiments, the antibody is BIIB059, anifrolumab, daxdilimab (VIB7734) or belimumab. In some embodiments, the fusion protein is tagraxofusperzs (Elzonris). In some embodiments, the corticosteroid is dexamethasone or prednisone.

In some embodiments, the treatment reduces production of inflammatory cytokines or chemokines by dendritic cells in the human subject.

In some embodiments, the inflammatory cytokines or chemokines are selected from the group consisting of: type I interferon (IFN-I), IL-6, or TNF-α, type III interferon, MIP-1a/CCL3, MIP-1/CCL4, CCL5/RANTES, and IP-10/CXCL10.

In some embodiments, the method inhibits and/or reduces IFN-I production in the human subject in need thereof by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the human subject or in a control.

In some embodiments, the treatment reduces the expression of one or more of the interferon stimulated genes selected from the group consisting of Guanylate Binding Protein 1 (GBP1), Interferon Regulatory Factor 7 (IRF7), interferon stimulated gene 54 (ISG54), myxovirus resistance protein B (MxB), and 2′-5′-Oligoadenylate Synthetase 2 (OAS2).

In some embodiments, the treatment enhances expression of phosphoglycerate dehydrogenase (PHGDH), phosphoserine Phosphatase (PSPH), and phosphoserine Aminotransferase 1 (PSAT1).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A shows gene expression level of IFNA quantified at 5 h and normalized to TLR9 agonist treatment.

FIG. 1B shows secreted IFN-α quantified after 13 h of culture. For FIGS. 1A and 1B, purified pDCs from Healthy Donors (HDs) were first cultured with medium only or with tunicamycin (TM at 3 μg/ml) or with thapsigargin (TG at 0.5 μM) for 3 h when the TLR9 agonist (CpG-C274 at 0.075 μM) was added to the culture.

FIGS. 1C and 1D show volcano plot comparing gene expression analyzed by RNA-seq of pDCs from HDs cultured for 8 h with the TLR9 agonist CpG-C274 versus medium (FIG. 1C) or with CpG-C274 and tunicamycin versus CpG-C274 alone (FIG. 1D). Patterns on all graphs indicate differentially expressed genes (DEG) and UPR+ER stress genes, IFN genes and ISGs as indicated.

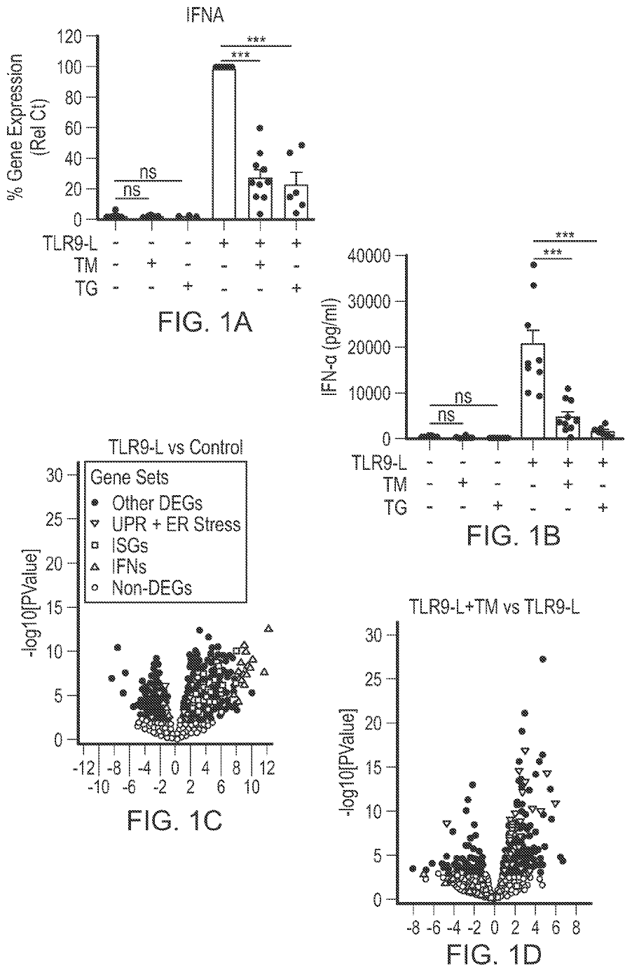

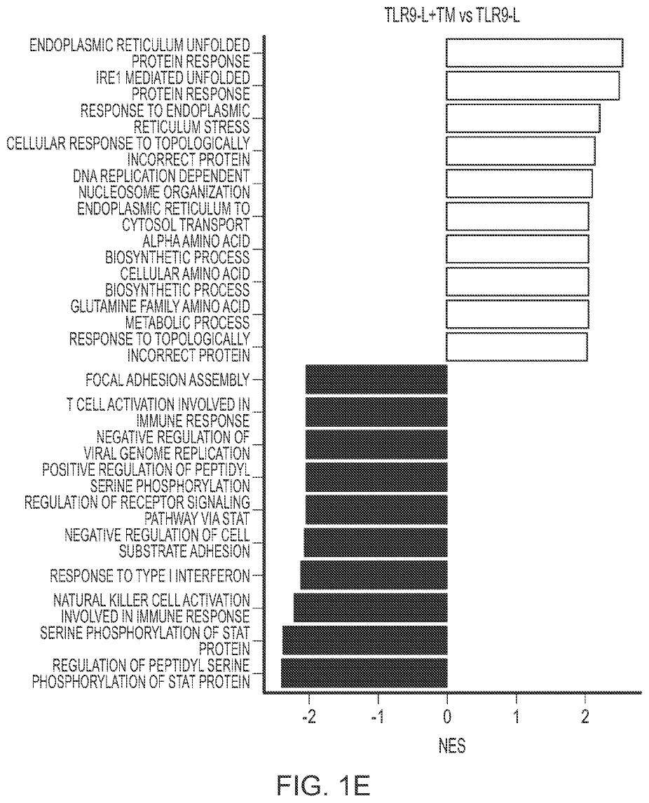

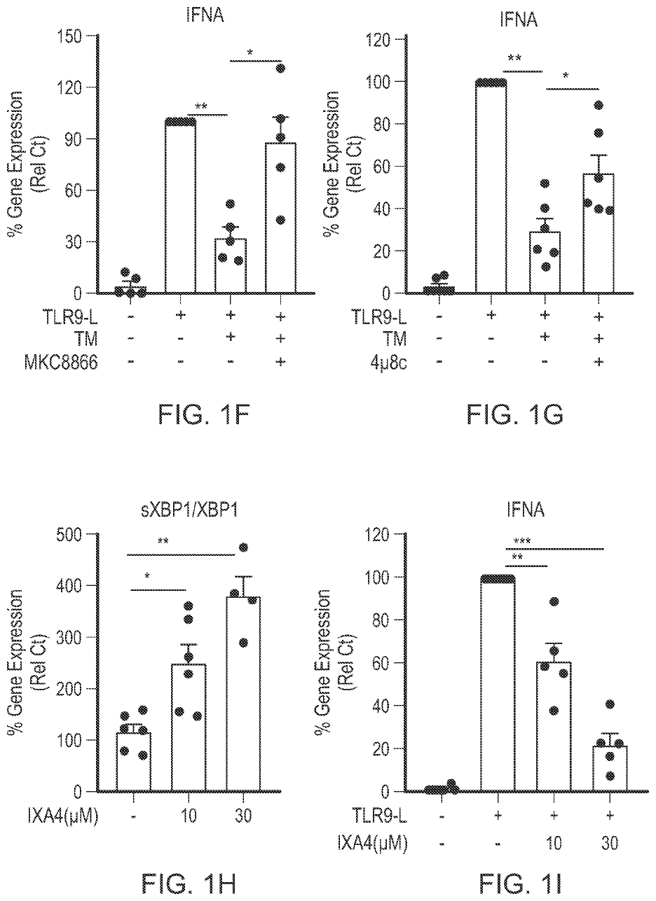

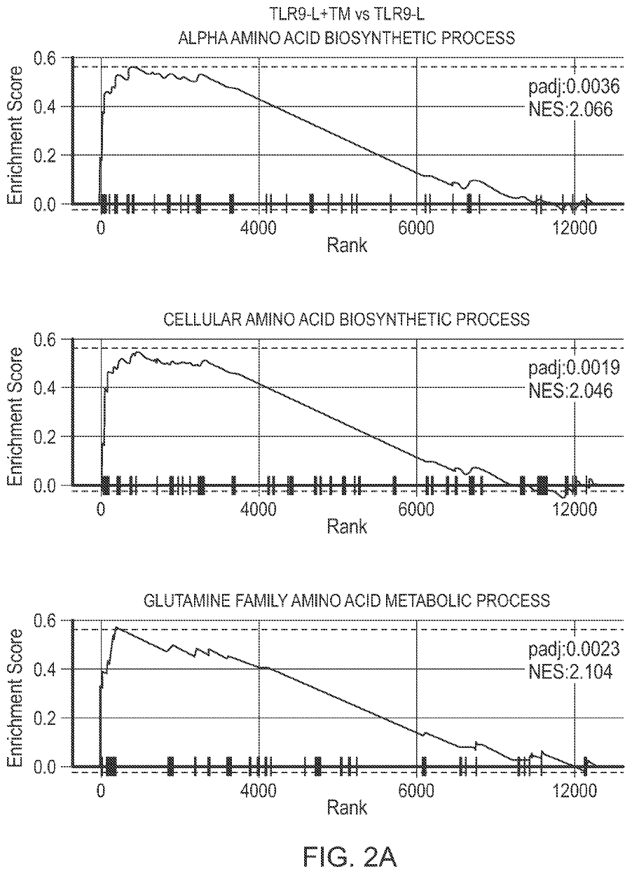

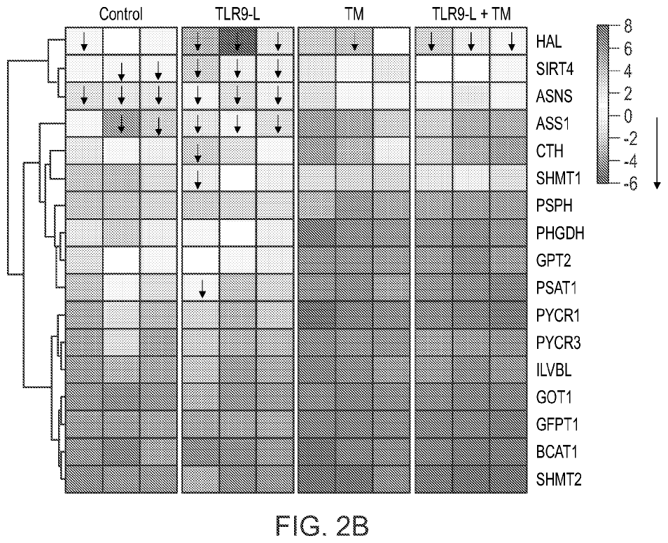

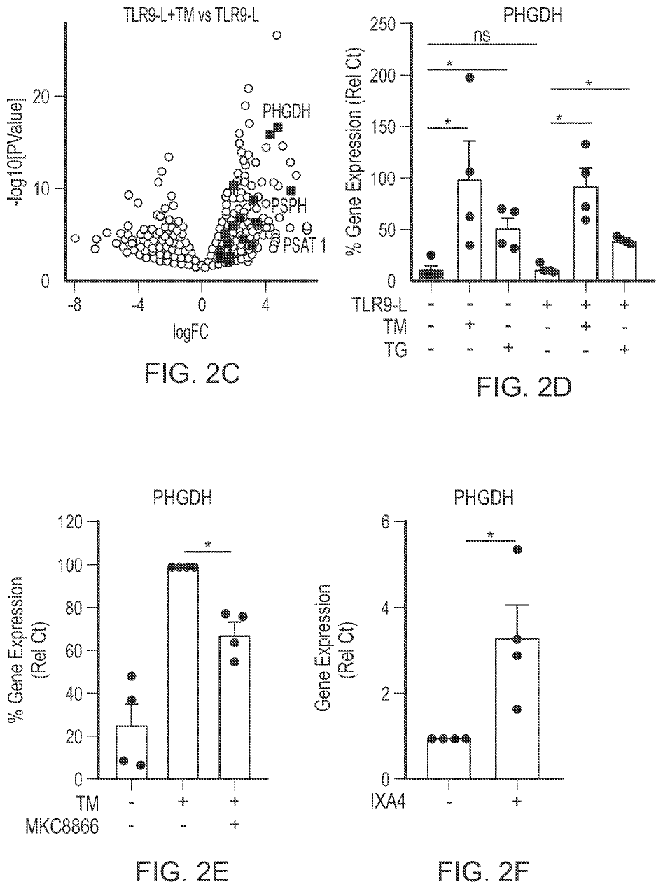

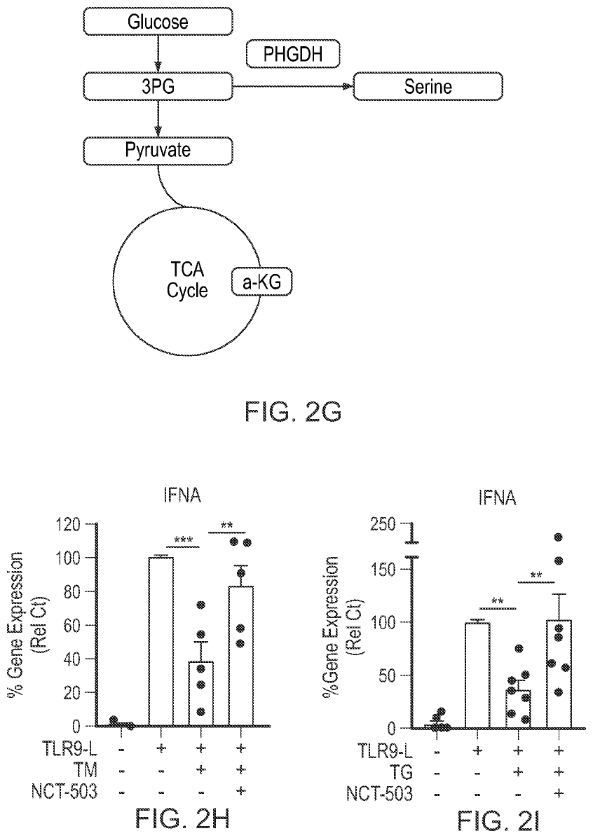

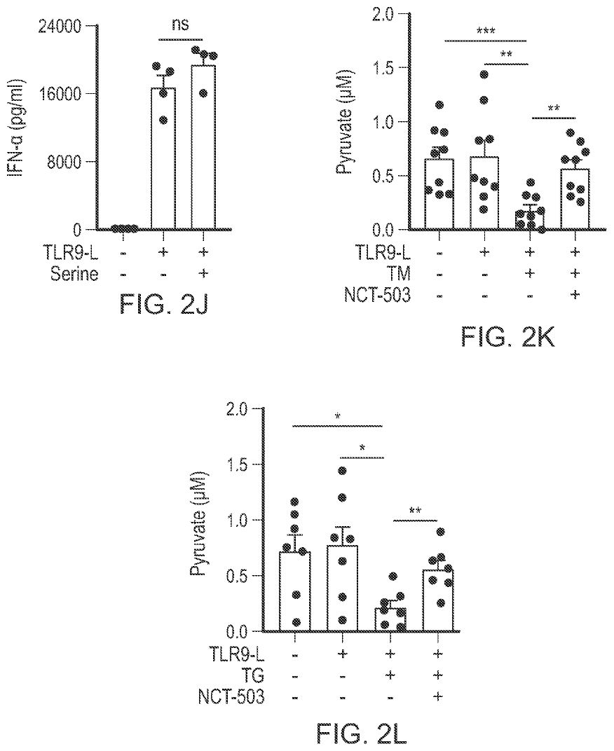

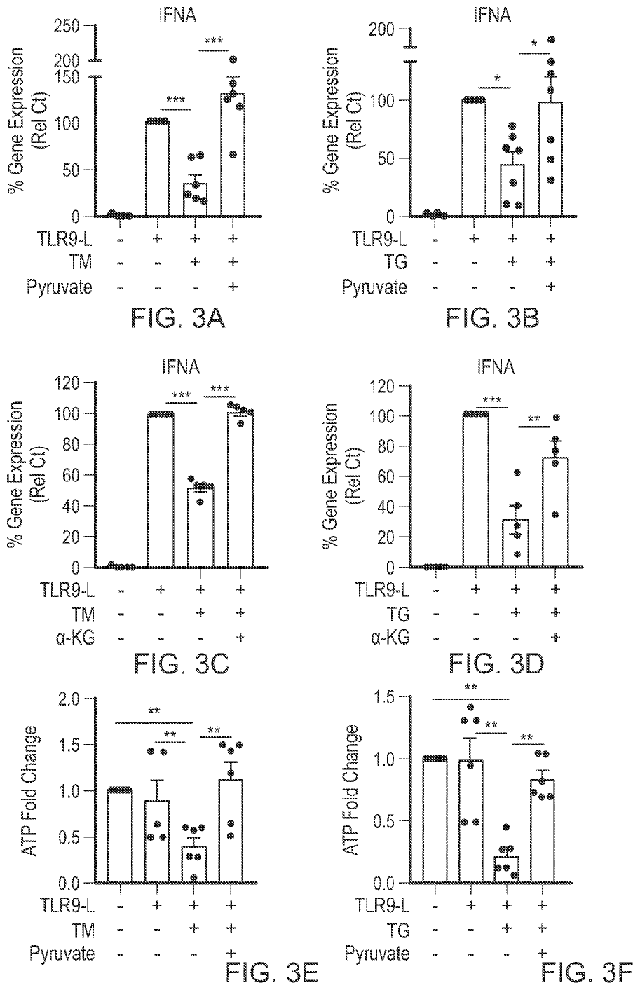

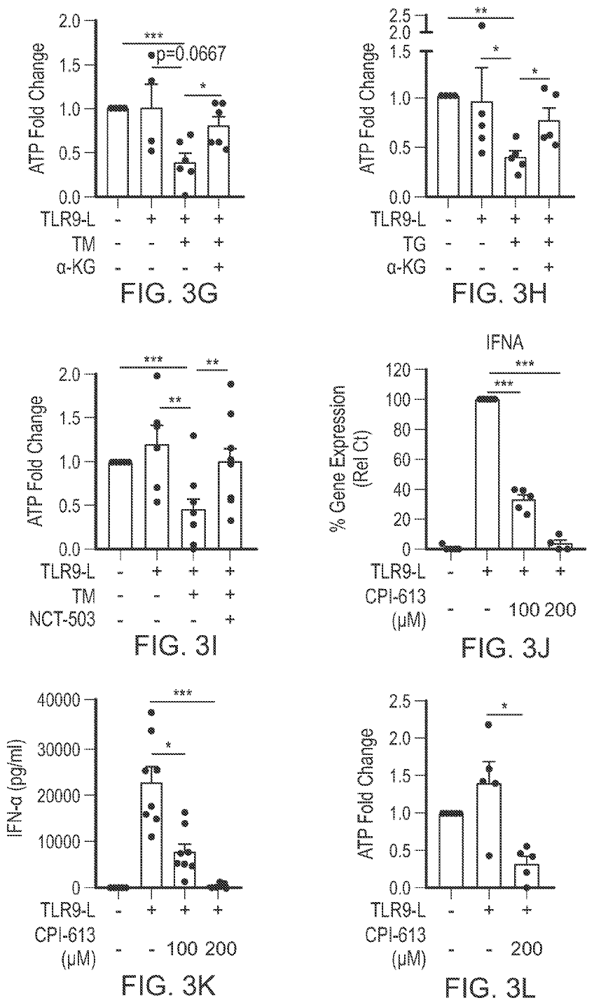

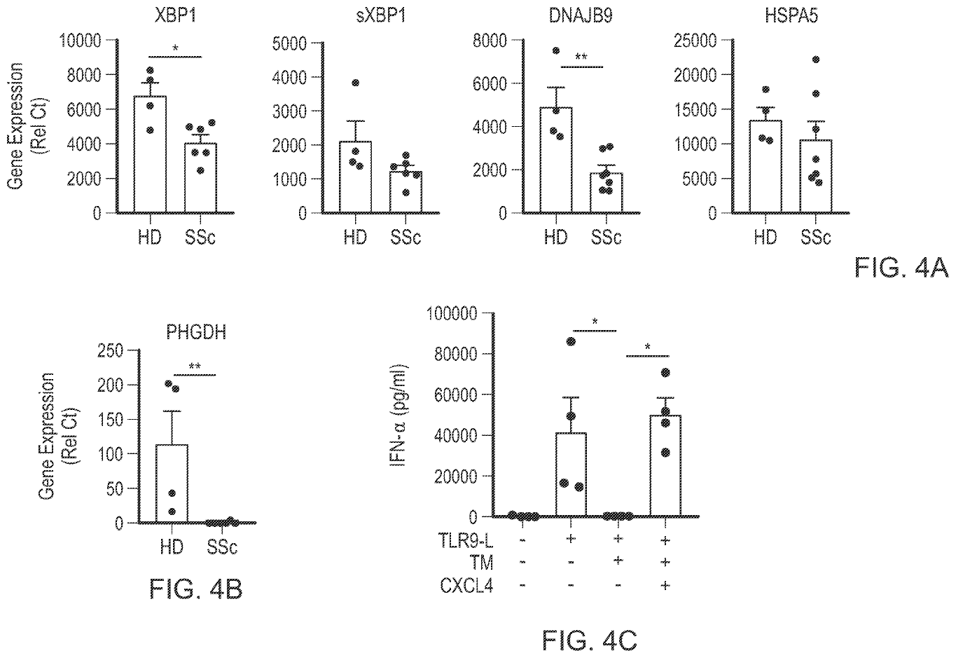

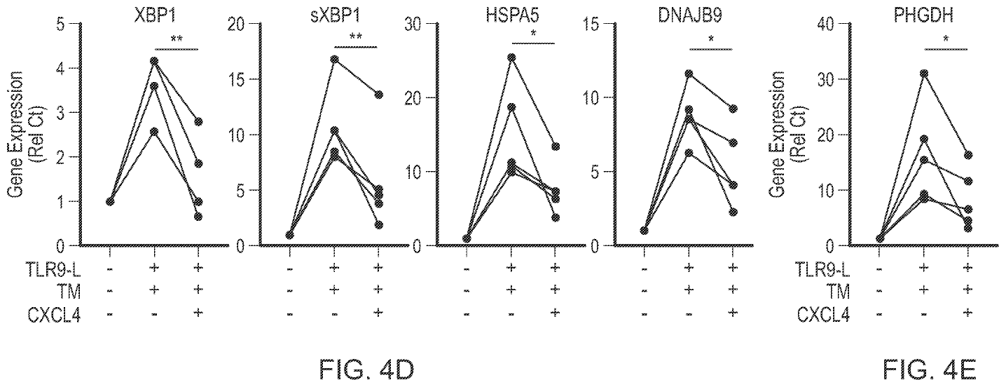

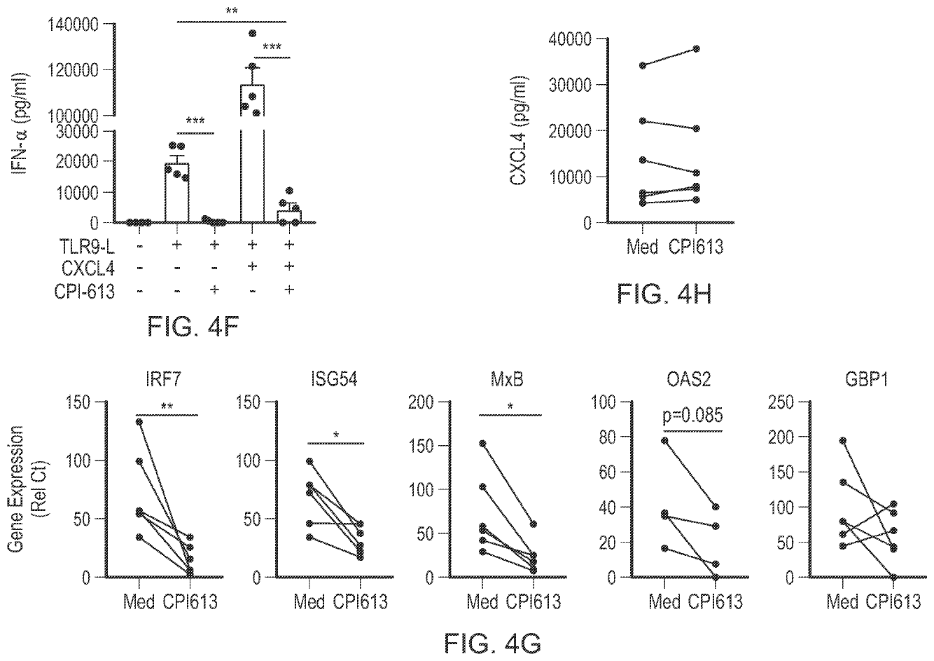

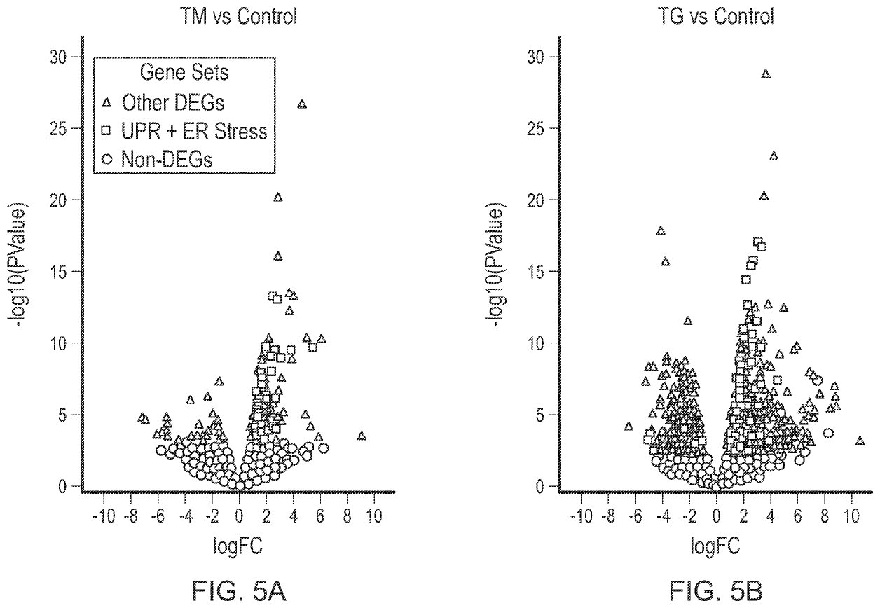

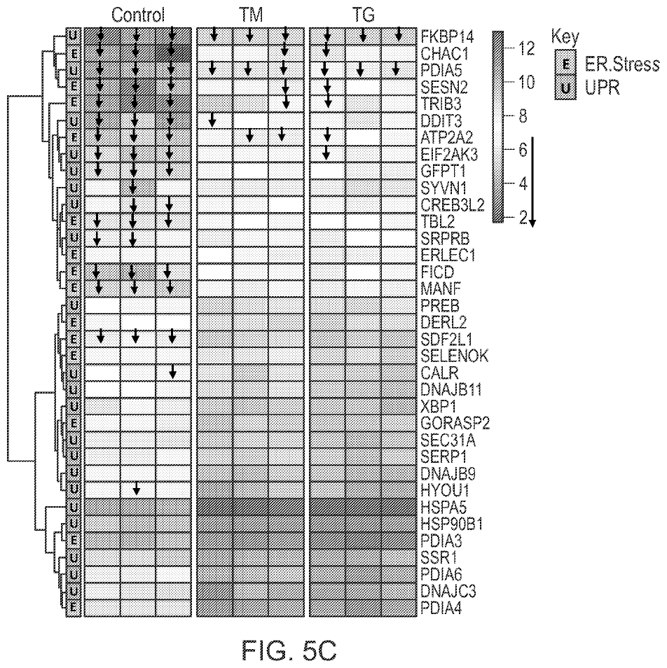

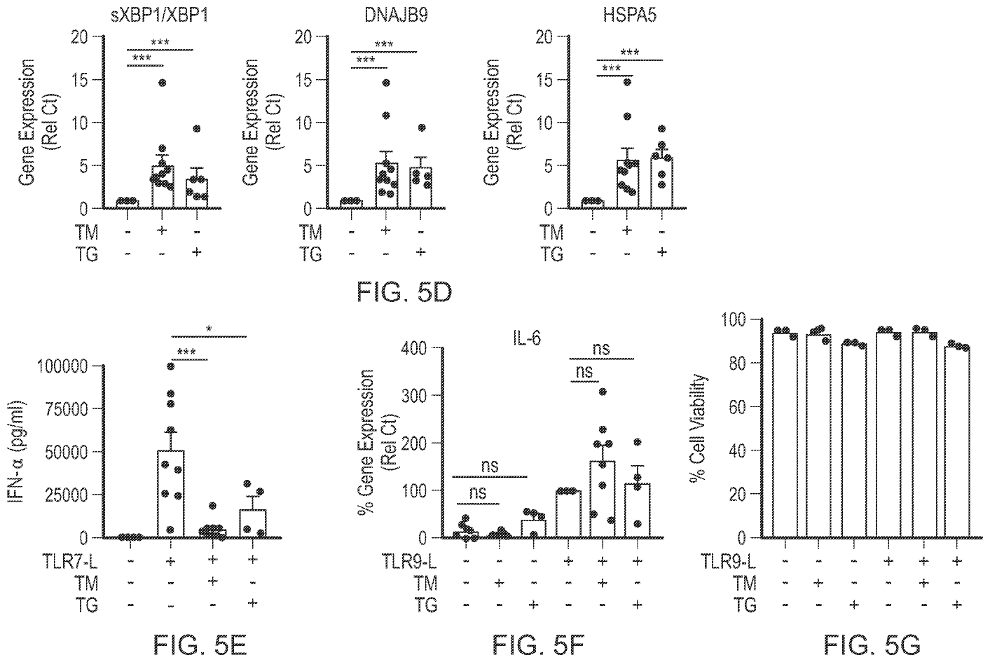

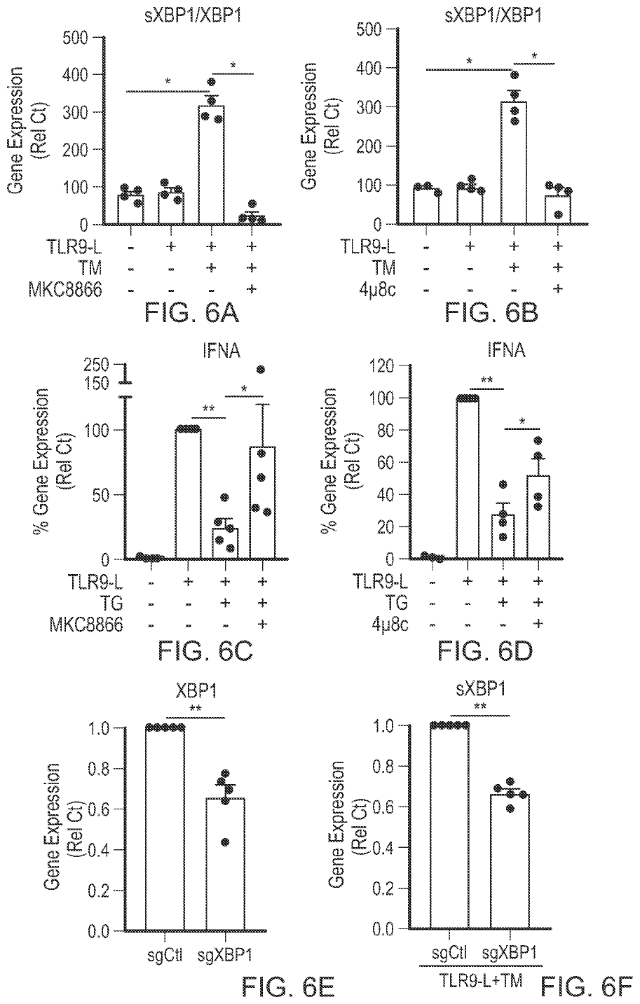

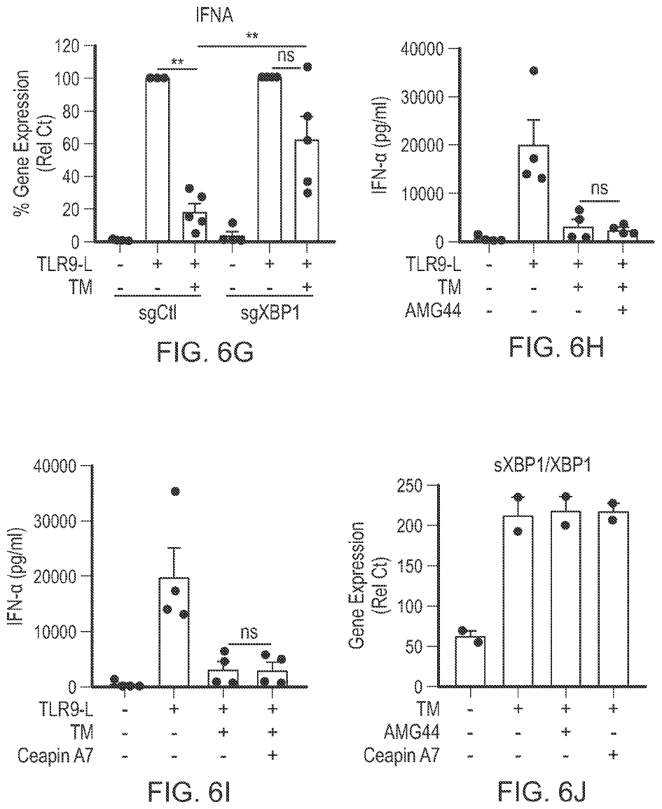

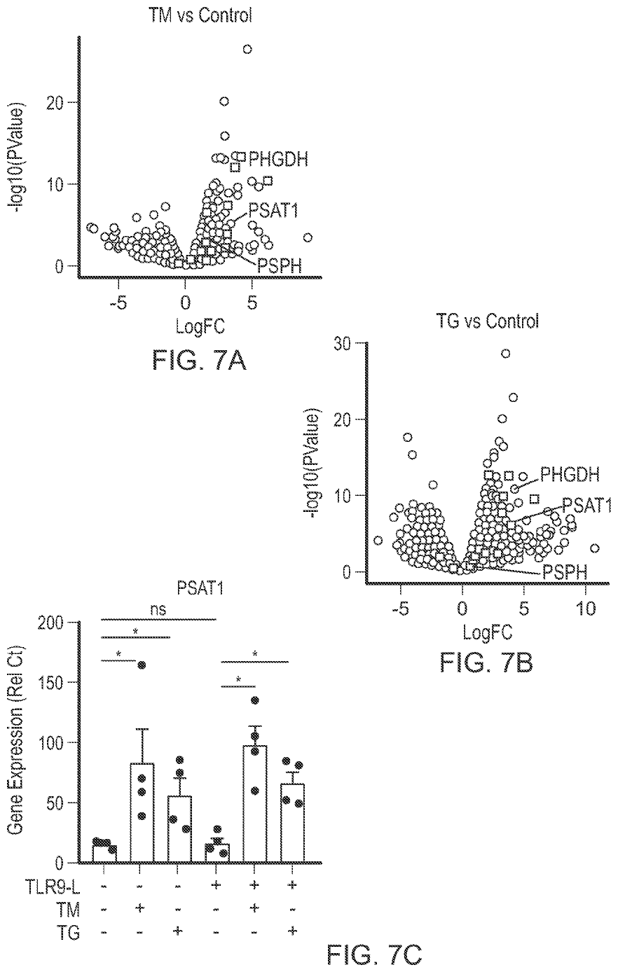

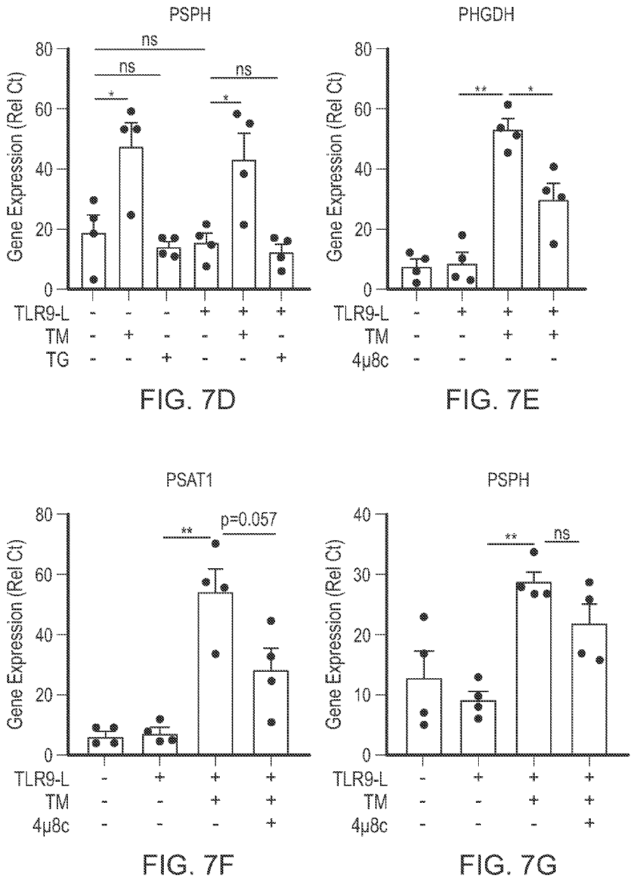

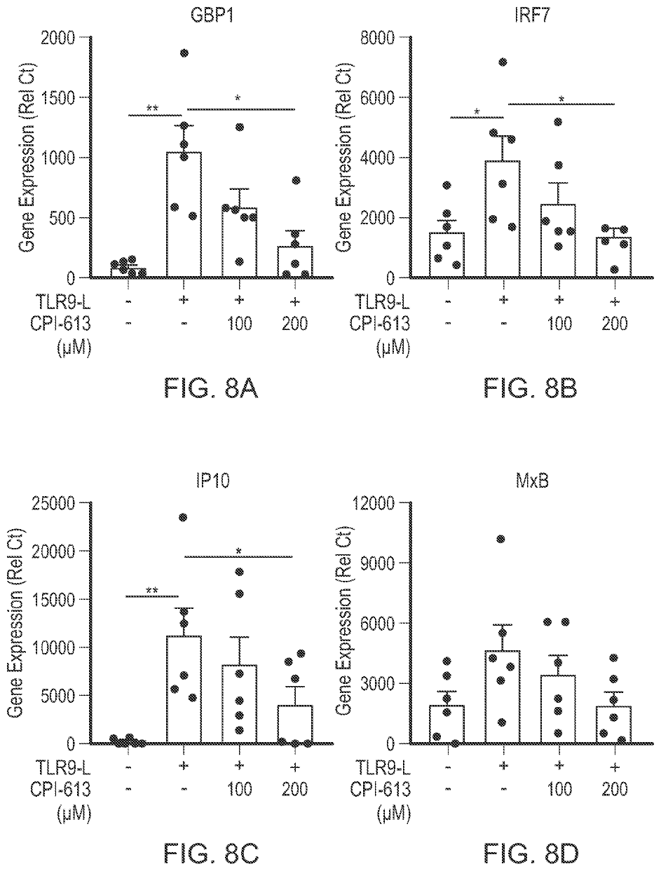

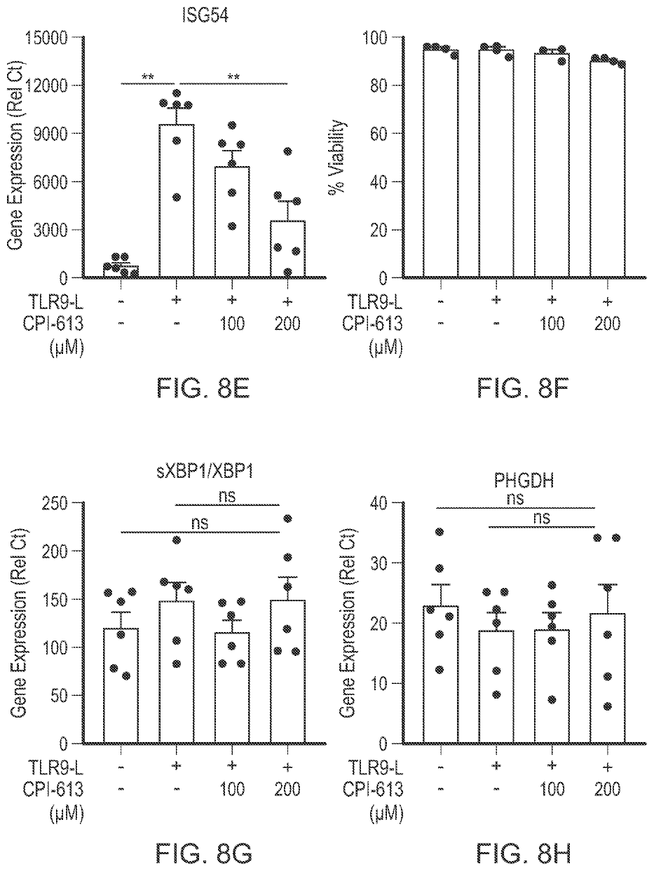

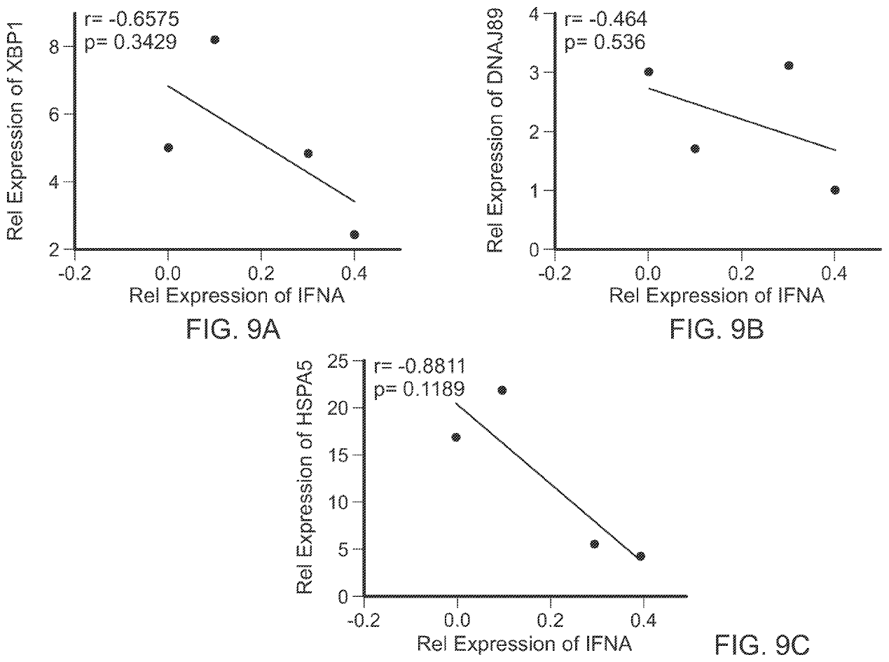

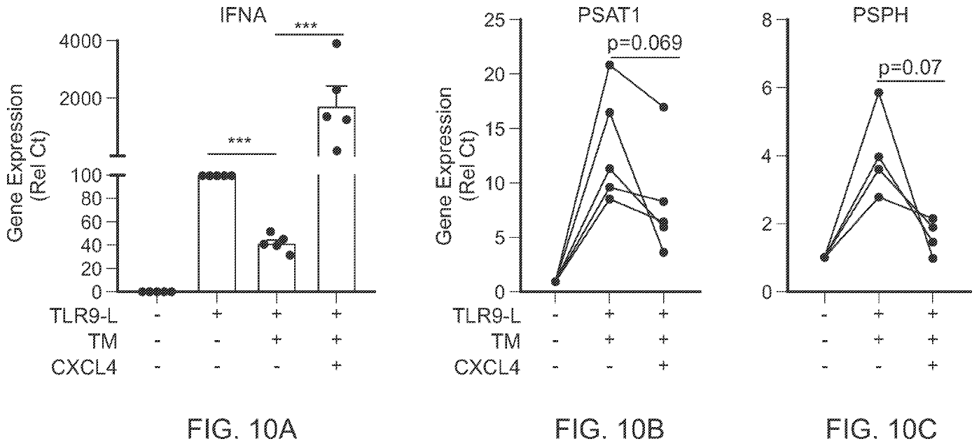

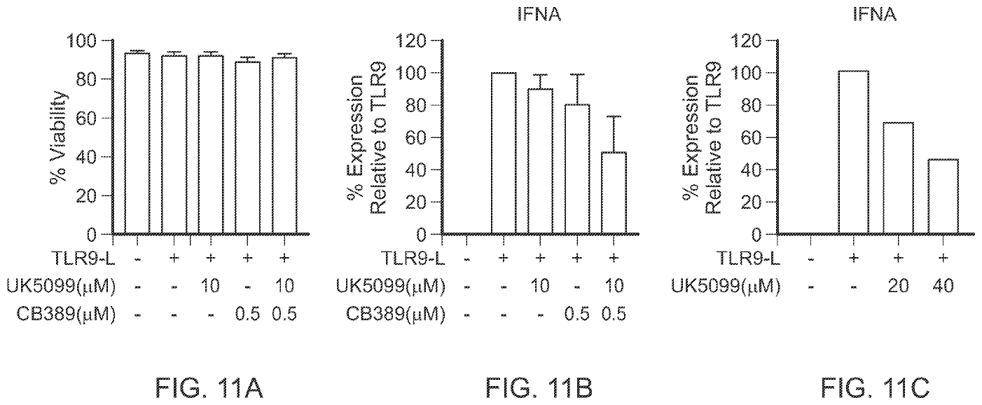

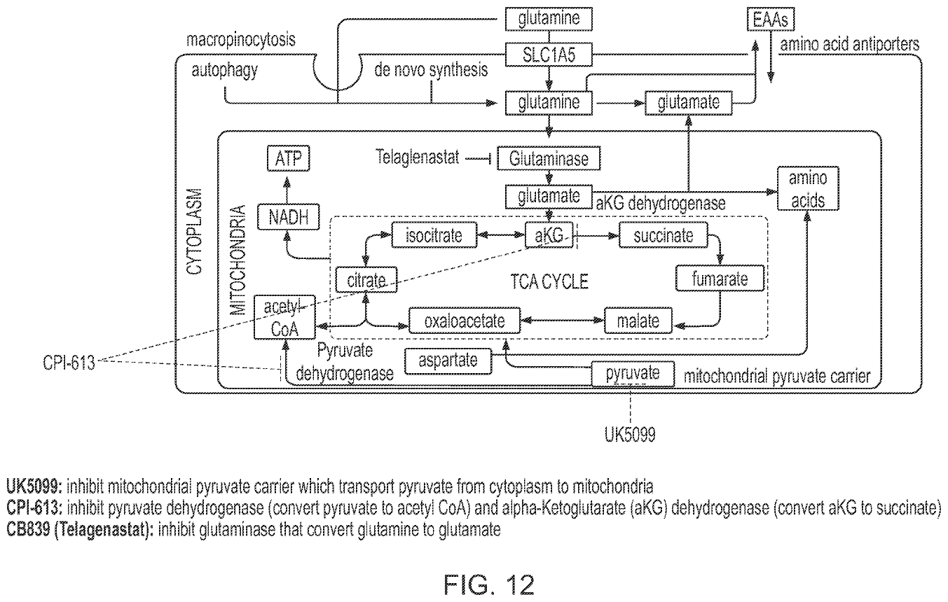

For FIG. 1E, all differentially expressed genes in RNA-seq of pDCs from HDs were cultured for 8 h with CpG-C274 and tunicamycin versus CpG-C274 alone were analyzed for pathway analysis using GSEA gene enrichment analysis.

For FIG. 1F and FIG. 1G, pDCs from HDs were cultured with or without tunicamycin (TM) in the presence or absence of IRE1α inhibitor (MKC8866 at 1 μM or 4μ8c at 10 μM) for 3 h when TLR9 agonist was added to the culture. Gene expression level of IFNA was quantified at 5 h and normalized to TLR9 agonist treatment.

For FIG. 1H, pDCs were cultured in medium only or with XBP1 agonist (IXA4 at 10 μM & 30 μM) for 6 h and XBP1 splicing quantified.

For FIG. 1I, pDCs were cultured in medium only or with IRE1α-XBP1 agonist (IXA4 at 10 μM & 30 μM) for 1 h when TLR9 agonist were added to the culture. IFNA gene expression was quantified at 5 h and normalized to TLR9 agonist treatment. Individual donors are indicated, and all results are represented as a mean±SEM and statistical significance was evaluated using a Mann-Whitney U-test. *p<0.05, **p<0.01, ***p<0.001.

FIG. 2A shows the gene enrichment score of amino acid biosynthesis.

FIG. 2B shows heatmaps of genes involved in in amino acid biosynthesis. Arrows indicate genes that have low expression (0 to −6 represents expression of less than 1 count per million with −6 representing log 2 (−6) count per million); no arrows indicate genes with expression of more than 1 count per million with 8 representing log 2 (8) count per million.

FIG. 2C shows Volcano plot comparing gene expression of serine biosynthesis analyzed by RNA-seq of pDCs from HDs cultured for 8 h with TLR9 agonist and tunicamycin vs TLR9 agonist alone. For FIGS. 2A-2C, purified pDCs from HDs were cultured in medium or with tunicamycin (TM) for 3 h, followed by TLR9 agonist for 5 h.

FIG. 2D shows gene expression of PHGDH when pDCs were cultured in media alone or with tunicamycin or thapsigargin for 3 h, followed by TLR9 agonist for 5 h.

FIG. 2E shows gene expression level of PHGDH quantified and normalized to tunicamycin treatment when pDCs were cultured with tunicamycin alone or in combination with tunicamycin and IRE1α inhibitor (MKC8866 at 1 μM) for 8 h. FIG. 2F shows gene expression level of PHGDH quantified and normalized to medium when pDCs were cultured in medium or with IRE1α-XBP1 agonist (IXA4 at 30 μM) for 6 h.

FIG. 2G shows the graphical representation of the role of PHGDH in glucose metabolism. FIGS. 2H and 2I show gene expression level of IFNA quantified at 5 h and normalized to TLR9 agonist treatment. pDCs were cultured in medium or with tunicamycin (TM) or thapsigargin (TG) in combination with PHGDH inhibitor (NCT-503 at 2 μM) for 3 h, when TLR9 agonist was added to culture. FIG. 2J shows secreted IFN-α was quantified by ELISA after 13 h of culture. pDCs were cultured in medium or with L-serine (1 mg/ml) for 1 h when TLR9 agonist was added to culture. FIGS. 2K and 2L show intracellular pyruvate when pDCs were cultured with tunicamycin (TM) and thapsigargin (TG) in combination with PHGDH inhibitor (NCT-503 at 2 μM) for 3 h, and TLR9 agonist was added to the culture for 2.5 h. Individual donors are indicated, and all results are represented as a mean±SEM and statistical significance was evaluated using a Mann-Whitney U-test. *p<0.05, **p<0.01, ***p<0.001.

FIGS. 3A and 3B show gene expression level of IFNA quantified at 5 h and normalized to TLR9 agonist treatment. FIGS. 3C and 3D show gene expression level of IFNA quantified at 5 h and normalized to TLR9 agonist treatment. FIGS. 3E and 3F show intracellular ATP quantified using ATP assay kit after 2.5 h of culture and normalized to medium. FIGS. 3G and 3H show intracellular ATP quantified after 2.5 h of culture and normalized to medium. FIG. 3I shows intracellular ATP quantified after 2.5 h of culture and normalized to medium. pDCs were cultured in medium or with tunicamycin in combination with PHGDH inhibitor (NCT-503 at 2 μM) for 3 h, when TLR9 agonist was added to culture. FIG. 3J shows gene expression level of IFNA quantified at 5 h and normalized to TLR9 agonist treatment. FIG. 3K shows secreted IFN-α quantified by ELISA after 13 h of culture. FIG. 3L shows intracellular ATP quantified after 4 h of culture and normalized to medium. For FIGS. 3A, 3B, 3E, and 3F, pDCs were cultured with tunicamycin or thapsigargin alone or with sodium pyruvate (pyruvate at 10 mM) for 3 h, when TLR9 agonist was added to the culture. For FIGS. 3C, 3D, 3G, and 3H, pDCs were cultured with tunicamycin or thapsigargin alone or with α-ketoglutaric acid disodium salt (α-KG at 10 mM) for 3 h, when TLR9 agonist were added to the culture. For FIGS. 3J-3L, pDCs were cultured in medium or with inhibitor for PDH and α-KGDH (CPI-613 at 100 μM & 200 μM) for 1 h, when TLR9 agonist was added to the culture. Individual donors are indicated, and all results are represented as a mean±SEM and statistical significance was evaluated using a Mann-Whitney U test. *p<0.05, **p<0.01, ***p<0.001.

FIG. 4A shows gene expression of XBP1, spliced XBP1, HSPA5, DNAJB9. FIG. 4B shows gene expression of PHGDH. FIG. 4C shows secreted IFN-α quantified by ELISA after 13 h of culture. FIG. 4D shows gene expression level of XBP1, spliced XBP1, HSPA5, DNAJB9 after 5 h of culture. FIG. 4E shows gene expression level of PHGDH after 5 h of culture. FIG. 4F shows secreted IFN-α quantified by ELISA after 13 h of culture. FIG. 4G shows gene expression levels of the interferon stimulated genes GBP1, IRF7, ISG54, MxB, OAS2. FIG. 4H shows secreted CXCL4 quantified by ELISA. For FIGS. 4A and 4B, pDCs were isolated from freshly isolated blood of healthy donors and patients with SSc. RNA was collected and quantified for gene expression of the indicated genes. Individual donors are indicated, and all results are represented as a mean±SEM and statistical significance was evaluated using a two tailed unpaired t test. *p<0.05, **p<0.01. For FIGS. 4C-4H, pDCs were cultured in media alone or with tunicamycin (TM) for 3 h, when TLR9 agonist was added to culture with/without CXCL4 (3 μg/ml). Statistical significance was evaluated using two tailed pair t test. *p<0.05, **P<0.01. For FIGS. 4F-4H, pDCs were cultured in media alone or with inhibitor of PDH and α-KGDH (CPI-613 at 200 μM) for 1 h, when TLR9 agonist with/without CXCL4 were added to culture. Statistical significance was evaluated using a Mann-Whitney U test. *p<0.05, **p<0.01, ***p<0.001. pDCs were isolated from patients with SSc and cultured with CPI-613 at 200 μM for 13 h. Statistical significance was evaluated using a Mann-Whitney U test. *p<0.05, **p<0.01, ***p<0.001.

FIGS. 5A and 5B show volcano plots comparing gene expression analyzed by RNA-seq of human pDCs from HDs cultured for 8 h in media alone or with tunicamycin (FIG. 5A; TM at 3 μg/ml) or thapsigargin (FIG. 5B; TG at 0.5 μM). Patterns in all graphs indicate differentially expressed genes (DEG) and UPR+ER stress genes as indicated.

FIG. 5C shows heatmaps of genes involved in the ER stress pathway induced by tunicamycin and thapsigargin. Arrows indicate genes that have lower expression (5 or less represents expression of less than log 2 (5) count per million); no arrows indicate genes with higher expression of more than log 2 (5) count per million.

FIG. 5D shows gene expression level of sXBP1/XBP1, DNAJB9 and HSPA5 quantified by Q-PCR after of 8 h culture and normalized to medium. pDCs were cultured in medium or with tunicamycin (TM)/thapsigargin (TG).

FIG. 5E shows secreted IFN-α quantified by ELISA after 13 h of culture. pDCs were cultured in medium or with tunicamycin/thapsigargin for 3 h when the TLR7 agonist (influenza virus FLU at 0.5 pfu/cell) were added to culture.

FIG. 5F shows gene expression level of IL-6 quantified at 5 h and normalized to TLR9 agonist treatment. FIG. 5G shows cell viability quantified via flow cytometer. Individual donors are indicated, and all results are represented as a mean±SEM.

For FIGS. 5F and 5G, pDCs were cultured in medium only or with tunicamycin or with thapsigargin for 3 h when the TLR9 agonist were added to the culture. Statistical significance was evaluated using a Mann-Whitney U-test; p>0.05, *p<0.05, ***p<0.001.

FIGS. 6A and 6B show Gene expression levels of the sliced IBP1 isoform quantified at 5 h by Q-PCR. For FIGS. 6A-6D, human pDCs were cultured in medium only or with tunicamycin (TM) or with thapsigargin (TG) in combination with IRE1α inhibitors (MKC8866 at 1 μM or 4μ8c at 10 μM) for 3 h when a TLR9 agonist were added to the culture. FIGS. 6C and 6D show gene expression levels of IFNA quantified at 5 h by Q-PCR and normalized to TLR9 agonist treatment. Statistical significance was evaluated using two tailed pair t test. *p<0.05, **p<0.01. For FIGS. 6E-6G, pDCs were electroporated with Cas9-sgRNA complex targeting XBP1 and cultured with IL-3 (20 ng/ml) for 72 h. Tunicamycin was added to culture for 3 h, followed by TLR9 agonist (CpG-C274 at 0.3 μM) for 5 h. FIGS. 6E and 6F show gene expression levels of XBP1 and IBP1 isoforms respectively. FIG. 6G shows IFNA quantified by Q-PCR. IFNA expression was normalized to TLR9 agonist treatment. FIGS. 6H and 6I show secreted IFN-α quantified by ELISA after 13 h of culture. pDCs were cultured in medium or with tunicamycin either alone or with a PERK inhibitor (FIG. 6H; AMG44 at 1 PM) or an ATF6 inhibitor (FIG. 6I; Ceapin A7 at 5 μM) for 3 h, when the TLR9 agonist were added to culture. FIG. 6J shows the expression of the XBP1 isoforms quantified by Q-PCR. pDCs were cultured in medium or with tunicamycin either alone or with AMG44 or Ceapin A7 for 8 h. Statistical significance was evaluated using a Mann-Whitney U test. *p<0.05, **p<0.01.

FIGS. 7A and 7B show volcano plots comparing gene expression analyzed by RNA-seq of pDCs cultured for 8 h with tunicamycin (TM) or thapsigargin (TG) vs medium. FIGS. 7C and 7D show gene expression of PSAT1, and PSPH quantified by Q-PCR. pDCs were cultured in media alone or with tunicamycin or thapsigargin for 3 h, followed by TLR9 agonist for 5 h. FIGS. 7E-7G show gene expression of PHGDH, PSAT1 and PSPH quantified by Q-PCR. FIG. 7E-7G, pDCs were cultured in media alone or with tunicamycin either alone or in combination with IRE1α inhibitors (4μ8c at 10 μM) for 3 h, followed by a TLR9 agonist for 5 h. Individual donors are indicated, and all results are represented as a mean±SEM and statistical significance was evaluated using a Mann-Whitney U-test and; ns p>0.05, *p<0.05, **p<0.01

FIGS. 8A-8E show gene expression level of the ISGs GBP1, IRF7, CXCL10, MxB, ISG54. FIG. 8F shows cell viability quantified by flow cytometer. FIGS. 8G and 8H show gene expression level of spliced XBP1 and of PHGDH quantified by Q-PCR. For FIGS. 8A-8H, human pDCs were cultured in medium or with an inhibitor for both PDH and α-KGDH (CPI-613 at 100 & 200 μM) for 1 h, when TLR9 agonist was added to the culture for 5 h. Individual donors are indicated, and all results are represented as a mean±SEM and statistical significance was evaluated using a Mann-Whitney U test. ns p>0.05, *p<0.05, **p<0.01

FIGS. 9A-9C show relative expression of IBP1 and IFN-α (FIG. 9A), DNAJB9 and IFN-α, (FIG. 9B) and HSPA5 and IFN-α (FIG. 9C). pDCs were isolated from the blood of patients with SSc. RNA was collected and quantified for gene expression of IFN-α, DNAJB9, HSPA5 and XABP1. Correlation co-efficient were calculated between the indicated genes.

FIG. 10A shows gene expression level of IFNA, quantified after 5 h of culture by Q-PCR. FIG. 10B shows gene expression level of PSAT1, quantified after 5 h of culture by Q-PCR. FIG. 10C shows gene expression level of PSPH, quantified after 5 h of culture by Q-PCR. Human pDCs were cultured in media alone or with tunicamycin (TM) for 3 h, when a TLR9 agonist was added to the culture either alone or with CXCL4 (3 μg/ml). Statistical significance was evaluated using two tailed pair t test. *<0.05, **P<0.01, ***P<0.001.

FIGS. 11A-11C show % viability and gene expression level of IFNA. FIG. 11A shows cell viability quantified at 5 h by flow cytometer. FIGS. 11B and 11C show % gene expression level of IFNA quantified at 5 h and normalized to TLR9 agonist treatment. Human pDCs from healthy donors were first cultured with medium only or with inhibitor for pyruvate transporter (UK5099 at 10, 20, and 40 μg/ml) or with inhibitor glutaminase (CB839 at 0.5 μM) for 1 h when the TLR9 agonist (CpG-C274 at 0.075 μM) was added to the culture.

FIG. 12 shows a schematic of the tricarboxylic acid cycle (TCA) and exemplary inhibitors of various molecules within the TCA cycle.

### DETAILED DESCRIPTION

This disclosure is based, in part, on the findings that the Inositol-Requiring Enzyme-X-Box Binding Protein 1 (IRE1α-XBP1 branch of the unfolded protein response (UPR)) inhibits the production of IFN-I by toll-like receptor (TLR)-activated plasmacytoid dendritic cells (pDCs). Mechanistically, IRE1α-XBP1 activation reprograms glycolysis to serine metabolism by inducing phosphoglycerate dehydrogenase (PHGDH) expression. This reduces pyruvate access into the tricarboxylic (TCA) cycle and blunts mitochondrial ATP generation that is necessary for IFN-I production. Furthermore, decreased expression of PHGDH and UPR-controlled genes in pDCs purified from patients with systemic sclerosis (SSc) was observed. Accordingly, pharmacological blockade of tri-carboxylic acid (TCA) cycle reactions inhibited IFN-I responses in pDCs of patients with SSc. These findings link the UPR to metabolic control of pDC hyperactivation and suggest that modulating this process may represent an unconventional strategy for the treatment of autoimmune diseases (such as SSc). The cover sheet U.S. Provisional Patent Application 63/121,133 filed Dec. 3, 2020 is incorporated by reference in its entirety.

Thus, this disclosure features agents that activate the UPR response in immune cells such as dendritic cells, and agents that disrupt the TCA cycle in such cells. The disclosure features methods of using such agents to treat a human subject with an autoimmune disease and/or to reduce production of inflammatory cytokines or chemokines by immune cells such as DCs (e.g., type I interferon (IFN-I), IL-6, or TNF-α, type III interferon, MIP-1a/CCL3, MIP-1/CCL4, CCL5/RANTES, and IP-10/CXCL10).

The methods of the disclosure can also be used to enhance expression of phosphoglycerate dehydrogenase (PHGDH), phosphoserine Phosphatase (PSPH), and phosphoserine Aminotransferase 1 (PSAT1) and reduce CXCL4 expression in immune cells, such as DCs.

A detailed description of the UPR activating agents and the TCA cycle disrupting agents, as well as methods of using these agents are set forth below.

### Unfolded Protein Response Activating Agents

The unfolded protein response (UPR) is an adaptive response that maintains the fidelity of the cellular proteome in conditions that subvert the folding capacity of the cell, such as those noticed in infection and inflammatory contexts. In immunity, the UPR sensor IRE1 (Inositol-requiring enzyme 1-alpha) is as a critical regulator of the homeostasis of antigen presenting cells (APCs). Flores-Santibáñez F, et al. Cells. 2019; 8(12):1563. The IRE1α/XBP1s signaling pathway is an arm of the unfolded protein response (UPR) that safeguards the fidelity of the cellular proteome during endoplasmic reticulum (ER) stress, and that has also emerged as a key regulator of dendritic cell (DC) homeostasis. Medel B. et al., Frontiers in Immunology, 2019(9); Article 3050.

In the context of this disclosure, compounds that activate the UPR in plasmocytoid dendritic cells, particularly the IRE1α/XBP1s signaling pathway, can be used in the methods to treat autoimmune conditions and/or to reduce proinflammatory cytokine production. Such UPR activating agents include, but are not limited to tunicamycin and thapsigargin. As described herein, the term “activates the UPR” refers to the ability of the agent to activate and/or enhance the unfolded protein response, in particular, the IRE1α-XBP1 signaling branch of the UPR in cells (e.g., immune cells such as macrophages, dendritic cells, T cells, B cells, etc).

Exemplary UPR activating agents that can be utilized in the methods described herein have the structures provided below:

TABLE 1
UPR activating agents and their structures

| Agent | Structure |
|---|---|
| Tunicamycin (NSC 177382) | 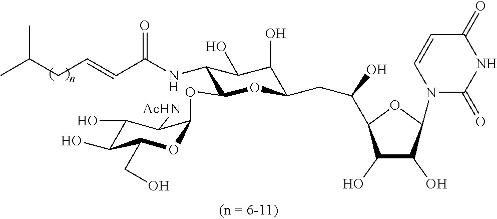 |
| Thapsigargin | 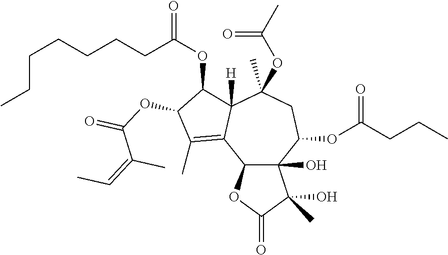 [SMILES: C/C=C(/C)C(=O)O[C@H]1C(C)=C2[C@H]([C@@H]1OC(=O)CCCCCCC)[C@@](C)(OC(C)=O)C[C@H](OC(=O)CCC)[C@@]1(O)[C@H]2OC(=O)[C@@]1(C)O] |
| IXA4 | 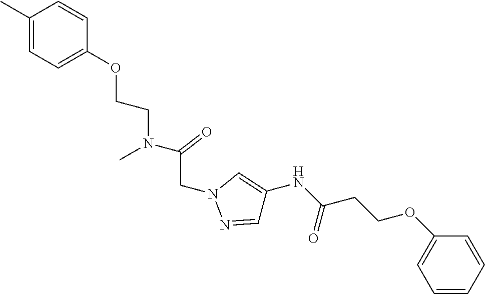 [SMILES: Cc1ccc(OCCN(C)C(=O)Cn2cc(NC(=O)CCOc3ccccc3)cn2)cc1] |

Any of the UPR activating agents shown in Table 1 or analogs thereof can be utilized in the methods of this disclosure.

### TCA Cycle Disrupting Agents

The tricarboxylic acid (TCA) cycle (also called the Krebs cycle) is the second stage of cellular respiration. It is a series of chemical reactions to release stored energy through the oxidation of acetyl-CoA derived from carbohydrates, fats, and proteins. In eukaryotic cells, the citric acid cycle occurs in the matrix of the mitochondrion. In the context of this disclosure, the term “disrupt”, with respect to the TCA cycle disrupting agents refers to agents that inhibit mitochondrial metabolism in cells such as immune cells (macrophages, dendritic cells, T cells, B cells, etc). The TCA cycle and exemplary inhibitors thereof are shown in FIG. 12.

In some embodiments, the TCA cycle disrupting agent is any of the compounds of Formula I or a pharmaceutically acceptable salt thereof as described in U.S. Pat. No. 9,839,691, incorporated by reference in its entirety. A compound of Formula I has the following structure:

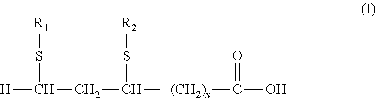

wherein R₁ and R₂ are independently selected from the group consisting of acyl defined as R₃C(0)-, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10. In some embodiments, R₁ and R₂ are benzyl or benzoyl. In some embodiments, the the compound of Formula I is

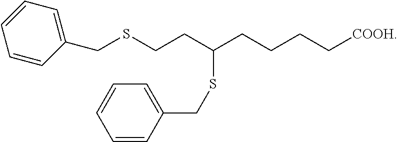 [SMILES: O=C(O)CCCCC(CCSCc1ccccc1)SCc1ccccc1]

In some embodiments, the compound of formula I is 6,8-bis-benzylthio-octanoic acid (CPI-913).

In some embodiments, the TCA cycle disrupting agent is UK5099 which inhibits mitochondrial pyruvate carrier, a carrier which transport pyruvate from cytoplasm to mitochondria. UK5099 has the following structure:

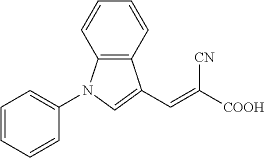 [SMILES: N#C/C(=C\c1cn(-c2ccccc2)c2ccccc12)C(=O)O]

In some embodiments, the TCA cycle disrupting agent is CB839 (Telagenastat), which inhibits glutaminase, an enzyme that converts glutamine to glutamate. CB839 has the following structure:

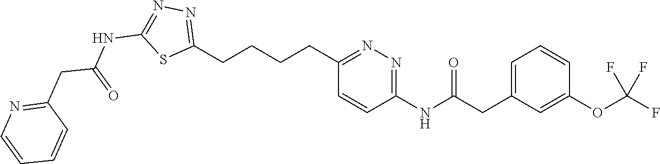 [SMILES: O=C(Cc1cccc(OC(F)(F)F)c1)Nc1ccc(CCCCc2nnc(NC(=O)Cc3ccccn3)s2)nn1]

### Autoimmune Diseases

The disclosure features methods of treating autoimmune diseases, in particular autoimmune rheumatic diseases, such as systemic sclerosis. Autoimmune rheumatic diseases are characterized by a breakdown of immune tolerance leading to inflammation and irreversible end-organ tissue damage. In some embodiments, the disclosed methods treat autoimmune diseases associated with dendritic cells (e.g., pDCs) or with IFN-I. See, e.g., Psarras A et al., Rheumatology, Volume 56, Issue 10, October 2017, Pages 1662-1675. In some embodiments, the disclosed methods treat autoimmune diseases associated with TNF, such as psoriatic arthritis, rheumatoid arthritis, ulcerative colitis, inflammatory bowel disease, and Crohn's disease. See, e.g., Jang D et al., Int. J. Mol. Sci. 2021, 22, 2719.

The pathogenesis of Systemic Sclerosis (SSc; scleroderma) is still unclear and remains elusive. However, scleroderma is a non-inherited, noninfectious disease and thought to be an autoimmune disease. SSc has a broad variety of symptoms triggered by excessive deposition of extracellular matrix in the dermis resulting in skin fibrosis. In later stages SSc is characterized by progressive tissue fibrosis affecting other internal organs as the gut, the lung or the kidneys. Therefore scleroderma is the hallmark of the disease comprising also e.g. lung fibrosis, renal fibrosis, fibrosis of the heart, the gut or the blood vessels. Inflammation, autoimmune disorders or vascular damage activates fibroblasts. Fibroproliferation is accompanied by excessive extracellular matrix production, dominated by Collagen type I resulting in progressive tissue fibrosis which can cause end organ failure and lead to high morbidity and mortality in patients with end-stage SSc (Harris et al. 2005—Kelley's Textbook of Rhematology 7th edition. Elsevier Saunders, Philadelphia PA).

The methods of this disclosure can be used to treat any type of systemic sclerosis, including Systemic Systemic Sclerosis (SSc), diffuse Systemic Sclerosis (dSSc), limited Systemic Sclerosis (ISSc), overlap type of Systemic Sclerosis, undifferentiated type of Systemic Sclerosis, malignant scleroderma, or Systemic Sclerosis sine scleroderma.

Apart from SSc, the methods of this disclosure can be used to treat a wide range of autoimmune rheumatic diseases, including, but not limited to systemic lupus erythematosus (SLE), rheumatoid arthritis (RA), Sjogren's syndrome, discoid lupus, cutaneous lupus, lupus nephritis, inflammatory bowel disease, psoriasis, type I diabetes, dermatomyositis, polymyositis and cutaneous autoimmune diseases (e.g., cutaneous lupus erythematosus, psoriasis, lichen planus, etc).

Psoriasis is an autoimmune disease that affects the skin. It occurs when the immune system mistakes the skin cells as a pathogen, and sends out faulty signals that speed up the growth cycle of skin cells. Psoriasis has been linked to an increased risk of stroke, and treating high blood lipid levels may lead to improvement. There are five types of psoriasis: plaque, guttate, inverse, pustular, and erythrodermic. The most common form, plaque psoriasis, is commonly seen as red and white hues of scaly patches appearing on the top first layer of the epidermis. However, some patients have no dermatological signs or symptoms.

Rheumatoid arthritis is a chronic inflammatory disorder that affects many tissues and organs, but principally attacks flexible joints. The process involves an inflammatory response of the capsule around the joints secondary to swelling of synovial cells, excess synovial fluid, and the development of fibrous tissue (pannus) in the synovium. The pathology of the disease process often leads to the destruction of articular cartilage and ankylosis of the joints.

Rheumatoid arthritis can also produce diffuse inflammation in the lungs, membrane around the heart (pericardium), the membranes of the lung (pleura), and white of the eye (sclera), and also nodular lesions, most common in subcutaneous tissue. Although the cause of rheumatoid arthritis is unknown, autoimmunity plays a pivotal role in both its chronicity and progression, and RA is considered a systemic autoimmune disease. Over expression of TNFa and other proinflammatory cytokines has been observed in patients with arthritis (Feldmann et. al, Prog Growth Factor Res., 4:247-55 (1992)). Furthermore, transgenic animals that over express human TNFa develop an erosive polyarthritis with many characteristics associated with the disease (Keffer et. al, EMBO J., 10(13):4025-31 (1991)). Analgesia and antiinflammatory drugs, including steroids, are used to suppress the symptoms, while disease-modifying antirheumatic drugs (DMARDs) are required to inhibit or halt the underlying immune process and prevent long-term damage. More recently, anti-TNFa antibody therapy (Rituximab) has been used to manage the disease (Edwards, et. al, N. Engl. J. Med., 350(25): 2572-81 (2004)).

Inflammatory bowel disease (IBD) is a group of inflammatory conditions of the colon and small intestine. The major types of IBD are Crohn's disease and ulcerative colitis (UC). The main difference between Crohn's disease and UC is the location and nature of the inflammatory changes: Crohn's disease can affect any part of the gastrointestinal tract, from mouth to anus (skip lesions), although a majority of the cases start in the terminal ileum; whereas, UC is restricted to the colon and the rectum. Depending on the level of severity, IBD may require immunosuppression to control the symptom, such as prednisone, TNF inhibition, azathioprine (Imuran), methotrexate, or 6-mercaptopurine. More commonly, treatment of IBD requires a form of mesalazine. Dermatomyositis (DM) is a type of autoimmune connective-tissue disease related to polymyositis (PM) that is characterized by inflammation of the muscles and the skin. While DM most frequently affects the skin and muscles, it is a systemic disorder that may also affect the joints, the esophagus, the lungs, and, less commonly, the heart.

Polymyositis (PM) (“inflammation of many muscles”) is a type of chronic inflammation of the muscles (inflammatory myopathy) related to dermatomyositis and inclusion body myositis.

Type I diabetes is a form of diabetes mellitus that results from autoimmune destruction of insulin-producing beta cells of the pancreas. The subsequent lack of insulin leads to increased blood and urine glucose. The classical symptoms are polyuria, polydipsia, polyphagia, and weight loss.

In some embodiments, the methods of the disclosure can treat autoimmune conditions which are IFN-I-mediated. For example, SLE is a prototypic IFN-I-mediated autoimmune disease whose clinical manifestations are diverse in the organs affected, severity and response to targeted and non-targeted therapies (Danchenko N, et al. Lupus 2006; 15:308-18.) For a review of the role of IFN-I in autoimmune diseases and current therapies, see Psarras A et al., Rheumatology, Volume 56, Issue 10, October 2017, Pages 1662-1675).

### Immune Cells

The methods of this disclosure may be used to block the TCA cycle and/or activate the UPR in a range of immune cells, including, but not limited to, dendritic cells, macrophages, T cells, B cells, natural killer cells, and/or neutrophils. Several types of immune cells that are involved in the pathology of autoimmune diseases. See, e.g., Anaya J-M et al., Front Immunol. 2016; 7: 139. Such immune cells are known to produce inflammatory cytokines or chemokines such as type I interferon (IFN-I), IL-6, or TNF-a, type III interferon, MIP-1a/CCL3, MIP-1/CCL4, CCL5/RANTES, and IP-10/CXCL10, that contribute to the pathology of autoimmune diseases.

Plasmocytoid dendritic cells (pDCs) are danger-sensing cells that produce interferon (IFN)-I. IFNs are generally classified into three families—IFN-I, IFN-II and IFN-III—which differ in their immunomodulatory properties, their structural homology and the group of cells from which they are secreted [3, 4]. IFN-Is (IFN-α, -β, -ω, -ε, -κ) compose the largest family and, alongside IFN-III (IFN-λ), activates intracellular signaling pathways that mediate immune responses against viruses and tumors. (Psarras A et al., Rheumatology, Volume 56, Issue 10, October 2017, Pages 1662-1675).

pDCs play a crucial role in antiviral immunity and have been implicated in the initiation and development of many autoimmune and inflammatory diseases, such as systemic lupus erythematosus (SLE) and systemic sclerosis (SSc). pDCs are regarded as precursor DC which are effectively interferon producing cells.

In some embodiments, the methods of the disclosure can be used to modulate signaling pathways in pDCs and other immune cells, thereby treating autoimmune conditions. In some embodiments, the pDCs that are modulated are dendritic cells express one or more of CD123, CD303 (BDCA2), CD304 (BDCA4), and immunoglobulin-like transcript 7 (ILT7), but do not express the lineage-associated markers (Lin) CD3, CD19, CD14, CD16 and CD11c. See, e.g., Ye, Y, et al., Clinical & Translational Immunology (2020); 9: e1139; Reizes, B. Immunity. 2019 Jan. 15; 50(1):37-50; Barrat F. J. and Su L., J Exp Med. 2019 Sep. 2; 216(9):1974-1985 for a review of pDCs, characterization of these cells, and their role in various autoimmune conditions.

### Additional Treatments

This disclosure features combination therapies wherein the UPR activating agent and/or the TCA cycle disruptor is administered with one or more additional treatments. The additional treatment can be an art-recognized therapy for autoimmune diseases (e.g., systemic sclerosis). See (Immunotherapies for autoimmune diseases. Nat Biomed Eng 3, 247 (2019)) and Elkhalifa S et al. (2018). Autoimmune Disease: Treatment. 10.1002/9780470015902.a0001437.pub3; Furie et al., J Clin Invest. 2019 Mar. 1; 129(3): 1359-1371) for a review of treatments, including immunotherapies, that can be used for autoimmune diseases. Such treatments include, but are not limited to the following: a nonsteroidal anti-inflammatory drug (NSAID), a fusion protein, an immunosuppressant, a corticosteroid, an anti-inflammatory cytokine an antimalarial and an antibody. The immunosuppressant that can be used as an additional treatment includes, but is not limited to, is methotrexate, mycophenolate mofetil (MMF), cyclophosphamide, cyclosporin, or azathioprine. The antimalarial that can be used as an additional treatment includes, but is not limited to, hydroxychloroquine or chloroquine. The antibody that can be used as an additional treatment includes, but is not limited to, BIIB059, anifrolumab, daxdilimab (VIB7734), or belimumab. The corticosteroid that can be used as an additional treatment includes, but is not limited to, dexamethasone, prednisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof. The fusion protein that can be used as an additional treatment includes, but is not limited to, tagraxofusperzs (Elzonris).

The anti-inflammatory cytokine that can be used as an additional treatment includes, but is not limited to, interleukin (IL)-1 receptor antagonist, IL-4, IL-6, IL-10, IL-11, and IL-13. Specific cytokine receptors for IL-1, tumor necrosis factor-alpha, and IL-18 also function as pro-inflammatory cytokine inhibitors. The nature of anti-inflammatory cytokines and soluble cytokine receptors are known in the art and discussed in Opal and DePalo, Chest, 117(4): 1162-72 (2000).

In some embodiments, the additional therapy includes one or more of: sulfasalazine, doxycycline, minocycline, penicillamine, tofacitinib, and leflunomide.

The components of the combination therapy may be administered substantially at the same time or sequentially.

### Methods of Treatment

The disclosure features a variety of methods for treating an autoimmune disease or condition, in particular, an autoimmune rheumatic disease or condition (e.g., SSc and SLE) using the agents described herein.

As used herein, the term “treat” “treatment,” or “treating” a subject having an autoimmune condition, are used in connection with a given treatment for a given disorder, wherein at least one symptom of the disorder is alleviated, or ameliorated. The treatment may inhibit deterioration or worsening of a symptom of the disclosed conditions (e.g., SSc or SLE) or may cause the condition to develop more slowly and/or to a lesser degree (e.g., fewer symptoms in the subject) in the subject than it would have absent the treatment. A subject is treated with the methods of this disclosure, to improve a condition, symptom, or parameter associated with a disorder or to prevent progression or exacerbation of the disorder (including secondary damage caused by the disorder) to either a statistically significant degree or to a degree detectable to one skilled in the art. A subject who is at risk for, diagnosed with, or who has one of the autoimmune conditions of this disclosure (e.g., SSc or SLE) can be administered a compound of this disclosure (e.g., an agent that activates the UPR and/or an agent that disrupts the TCA cycle) in an amount and for a time to provide an overall therapeutic effect. A compound of this disclosure can be administered alone (monotherapy) or in combination with other agents (combination therapy), which agents are described in the “Additional treatments” section.

As used herein, the term “therapeutically effective amount” of an agent is an effective amount that may be determined by the effect of the administered agents or the combined effects of the agents (if more than one agent is used). The “therapeutically effective amount” of the agent of this disclosure is an amount that results in a reduction in the severity of disease symptoms, the frequency and length of periods without disease symptoms. Preferably, it results in prevention of dysfunction or disability due to an increase in disease or distress. For example, in the case of systemic sclerosis, a therapeutically effective amount can be, for example, one that prevents dermal fibrosis, skin lesions, alopecia, inflammation, skin thickening, collagen deposition, proteinuria, autoantibody production, and complement deposition, It is preferable to prevent further deterioration of physical symptoms associated with systemic sclerosis. A therapeutically effective amount is also preferred to prevent or delay the onset of systemic sclerosis, as may be desired when early or preliminary signs of disease are present. Similarly, delaying the chronic progression associated with systemic sclerosis is also desired. Clinical trials are utilized in the diagnosis of systemic sclerosis include chemistry, hematology, histopathology, serology and radiology measures. Thus, any clinical or biochemical test that monitors the above can be used to determine whether a particular treatment is in a therapeutically effective amount to treat systemic sclerosis. Those skilled in the art will be able to determine such amounts based on factors such as the size of the subject, the severity of the subject's symptoms, and the particular composition or route of administration chosen.

Severity, progression, response to treatment, and other clinical measures of systemic sclerosis symptoms typically include improved Rodnan skin score, Raynaud's Condition Score, Lung function test, assessment of patients using forced spirometry, right heart catheter hemodynamics, serum creatine measurements, blood pressure and total blood counts, and serum creatinine phosphokinase levels (eg, Furst, 2008, Rheumatology, 47: v29-v30 and Furst et al., 2007, J. of Rheumatology, 34: 5, 1194-1200).

The therapeutically effective amount of the agent may also vary according to factors such as the disease state, the age, sex, and weight of the individual, and the ability of the compound to elicit a desired response in the individual, e.g., to ameliorate at least one parameter of the condition or to ameliorate at least one symptom of the condition. A therapeutically effective amount is also an amount where the therapeutically beneficial effect exceeds any toxic or detrimental effect of the composition. A therapeutically effective amount of an agent of this disclosure (i.e., an effective dosage) includes milligram, microgram, nanogram, or picogram amounts of the agent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

The amounts and times of administration for combination therapies can be those that provide, e.g., an additive or a synergistic therapeutic effect. Further, the administration of the compound of this disclosure (e.g., a UPR activator and/or a TCA cycle inhibitor) can be used as a primary, e.g., first line treatment, or as a secondary treatment, e.g., for subjects who have an inadequate response to a previously administered therapy (i.e., a therapy other than one with a compound of this disclosure). In some embodiments, the combination therapy includes the use of a compound of this disclosure with one or more of the following agents: glucocorticoid, NSAID, prednisone, hydroxychloroquine, chloroquine, amodiaquine, pyrimethamine, proguanil, mefloquine, dapsone, primaquine, methotrexate, mycophenolate mofetil, azathioprine, thalidomide, cyclophosphamide, cyclosporine A, rapamycin, prostacyclin, phosphodiesterase inhibitor, endothelin antagonists, statin, ACE inhibitor, calcium channel blockers, and an anti-BDCA2 antibody.

As used herein, the term “control” refers to an age-matched subject that does not have or is not diagnosed with n autoimmune condition. In some embodiments, a control refers to an age-matched and sex-matched subject that is not treated with the method of this disclosure, or is treated with a placebo. In some embodiments, a control refers to a population average for the amount or degree of a particular parameter in a normal healthy population.

Treatment outcomes on autoimmune diseases can be measured using any of the routine assays and techniques known in the art, including but not limited to enzyme-linked immunosorbent assay (ELISA), multiplex cytokines assay (Aziz N. Immunopathol Dis Therap. 2015; 6(1-2):19-22), qualitative and quantitative polymerase chain reaction (PCR), and patient-reported outcome measures. Clinical outcomes can be measured using several clinical features such as those described in Touma, Zahi (Ed.) Outcome Measures and Metrics in Systemic Lupus Erythematosus; Pages 1-50.

The methods of the disclosure can reduce production of inflammatory cytokines or chemokines by immune cells (such as dendritic cells) in the human subject. In some embodiments, the methods of this disclosure reduce IFN-I production in the human subject in need thereof by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the human subject or in a control. The methods can also reduce the expression of interferon stimulated genes, including, but not limited to, Guanylate Binding Protein 1 (GBP1), Interferon Regulatory Factor 7 (IRF7), interferon stimulated gene 54 (ISG54), myxovirus resistance protein B (MxB), and 2′-5′-Oligoadenylate Synthetase 2 (OAS2). The methods can also enhance expression of phosphoglycerate dehydrogenase (PHGDH), phosphoserine Phosphatase (PSPH), and phosphoserine Aminotransferase 1 (PSAT1). In some cases, the methods of the disclosure can reduce CXCL4 expression in DCs.

The efficacy of the methods of this disclosure on clinical disease can be measured based on the clinical monitoring and scoring techniques known in the art and routinely used in the assessment of autoimmune disease. For instance, a clinical score known as a Systemic lupus erythematosus disease activity index (SLEDAI) is an indicator of SLE disease activity measured and evaluated within the last 10 days (Bombardier C, Gladman D D, Urowitz M B, Caron D, Chang C H and Committee on Prognosis Studies in SLE, “Derivation of the SLEDAI for Lupus Patients.”, Arthritis Rheum 35: 630-640, 1992). Disease activity under the SLEDAI scoring system can range from 0 to 105. The following categories of SLEDAI activity have been identified: no activity (SLEDAI=0); mild activity (SLEDAI=1-5); moderate activity (SLEDAI=6-10); high activity (SLEDAI=11-19); very high activity (SLEDAI=20 and above) (Griffiths et al., “Assessment of Patients with Systemic Lupus Erythematosus and the use of Lupus Disease Activity Indices)”).

The British Isles Lupus Assessment Group BILAG index is an activity index for SLE based on specific clinical signs in the results of eight organ systems: whole body, mucocutaneous, nerve, skeletal muscle, cardiovascular, respiratory, kidney, and blood. Scoring is based on the character system, but a weighted numerical score can also be assigned to each character, and a BILAG score can be calculated in the range of 0-72 (Griffiths et al., “Evaluation of patients with systemic lupus erythematosus” And the use of Lupus Disease Activity Indices (Assessment of Patients with Systemic Lupus Erythematosus and the use of Lupus Disease Activity Indices)).

Physician comprehensive evaluation (PGA) score is a comprehensive assessment of a patient's disease activity by a physician. Physician writing an assessment of the patient's overall disease activity on a 3-inch visual analog scale with anchors at 0 (none), 1 inch (mild), 2 inches (medium), and 3 inches (severe) Is implemented. The improvement is measured by the decrease in the PGA score from visit to visit.

### EXAMPLES

The practice of the methods and compositions of the disclosure employs, unless otherwise indicated, conventional techniques of molecular biology (including recombinant techniques), cell culture, immunology, cell biology, and biochemistry, which are well within the purview of the skilled artisan. Such techniques are explained in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The materials, reagents, and methods, further described below, are used in the following examples. The invention, as described in the following examples, do not limit the scope of the invention described in the claims.

### Materials & Methods

### Patients

Participants were recruited from the IRB-approved Hospital for Special Surgery Scleroderma Registry and provided written informed consent before enrollment. All patients fulfilled the 2013 ACR/EULAR Classification Criteria for systemic sclerosis (SSc) (40). Patients were categorized as having limited (lSSc) or diffuse subtype (early diffuse (edSSc) or late diffuse (ldSSc)) of SSc according to LeRoy (41). Disease duration was defined as the time from the first SSc related symptom apart from the Raynaud phenomenon and was classified as early if the disease duration was ≤2 years. The clinical and demographic characteristics of the patients are described in Table 1 and Table 2.

TABLE 1
Clinical and demographic characteristics of the patients

| | |
|---|---|
| Age-years, mean (SEM) | 45.8 (6.3) |
| Sex-n, % female | 4, 66.7% |
| Race-number, percentage | 5, 83.3% Caucasian |
| | 1, 16.7% Other |
| Disease Duration-years, mean (SEM) | 8.5 (2.2) |
| Scleroderma subtype-n, % diffuse, | 3, 50% |
| n, % limited | 3, 50% |
| MRSS-mean (SEM) | 11.7 (4.7) |
| Autoantibody-n, % scl70, | 3, 50% Scl70 |
| n, % RNA Polymerase 3, | 1, 16.7% POL3 |
| n, % centromere, | 2, 33.3% CENB |
| Interstitial lung disease present-n, % | 5, 83.3% |
| Pulmonary hypertension present-n, % | 1, 16.7% |

TABLE 2
Clinical and demographic characteristics of the 6
individual patients as well as the overall
description of the patient population is shown.

| Subject # | Age | Sex | Race | Ethnicity | Treatment |
|---|---|---|---|---|---|
| 261 | 47 | F | White | Not | none |
| | | | | Hispanic | |
| 346 | 47 | F | White | Hispanic | mycophenolate, |
| | | | | | prednisone |
| 347 | 32 | F | White | Not | mycophenolate, Rituxan, |
| | | | | Hispanic | Amlodipine |
| 348 | 36 | F | Other | Hispanic | mycophenolate |
| 247 | 38 | M | White | Hispanic | s/p stem cell transplant |
| | | | | | sildenafil, omeprazole |
| 189 | 75 | M | White | Hispanic | hydroxychloroquine, |
| | | | | | ambrisentan, tadalafil |

Purification and Culture of pDCs from Healthy Donors and SSc Patients
Enriched leukocytes were obtained from New York blood center (Long Island City, NY) under internal Institutional Review Board-approved protocols. PBMCs were prepared using Ficoll-Paque density gradient and pDCs were isolated using BDCA4+ positive selection (Miltenyi Biotech: 130-097-415) as previously described (42). pDCs were cultured at 40,000 cells (for HDs) or at 10,000-20,000 cells (for SSc patients) per well in a 96-round bottom plate and incubated at 37° C., 5% CO2 and 95% humidity. For TLR7 and TLR9 activation assay, pDCs were stimulated with heat-inactivated 2 MOI of H1N1 VR-95 influenza A virus (ATCC) and 0.075 μM of C274 (42) respectively.

In some culture conditions, cells were cultured with the tunicamycin (thermofisher: 654380), thapsigargin (Sigma: T9033), 4μ8c (EMD Millipore: 412512), MKC8866 (Medchem Express: HY-104040), IXA4 (Chembridge: 131171.1), AMG PERK44 (R&D: 5517), Ceapin-A7 (Sigma: SML2330), NCT-503 (Axon Medchem: 2623), L-serine (EMD Millipore: S4500), sodium pyruvate (Sigma: 8636), α-ketoglutaric acid disodium salt hydrate (Sigma: K3752), CPI-613 (Selleckchem: S2776), Anti-PF4 antibody (Abcam: ab9561), CXCL4 (Sigma: SRP3142).

### Cell Viability Using Flow Cytometry

After 6-8 h of cell culture, pDCs were washed in PBS and then resuspending in FACS buffer and stained with DAPI. Cells were acquired by a fluorescence activated cell sorter (FACS) and analysis was performed using FlowJo analysis software. The gating strategy for viable cells involved progressively measuring total cells without uptake of DAPI.

### RNA Extraction and RT-PCR

After 6 h-13 h of cell culture, pDCs were lysed for total RNA extraction using the Qiagen RNeasy Plus Mini Kit. Quantity of RNA was measured by Nanodrop, and high-capacity cDNA Reverse Transcription kit (Thermofisher) was used to generate cDNA. qPCR reactions were performed. Gene expression levels were calculated based on relative threshold cycle (Ct) values as described (43). This was done using the formula Relative Ct=100 or 1000×1.8^{(HSK-GENE)}, where HSK is the mean CT of duplicate housekeeping gene runs (Ubiquitin), GENE is the mean CT of duplicate runs of the gene of interest, and 100/1000 is arbitrarily chosen as a factor to bring all values above 0. Primers are shown in Table 3.

TABLE 3
List of primers used in study

| Genes | SEQ ID NO | Sequences |
|---|---|---|
| h-IRF7-F | 1 | CTGTTTCCGCGTGCCCT |
| h-IRF7-R | 2 | GCCACAGCCCAGGCCTT |
| h-MxB-F | 3 | GAGACATCGGACTGCAGAT |
| h-MxB-R | 4 | GTGGTGGCAATGTCCACGTTA |
| h-ISG54-F | 5 | CTGGACTGGCAATAGCAAGCT |
| h-ISG54-R | 6 | AGAGGGTCAATGGCGTTCTG |
| h-GBP1-F | 7 | TGGAACGTGTGAAAGCTGAGTCT |
| h-GBP1-R | 8 | CATCTGCTCATTCTTTCTTTGCA |
| h-Ubiquitin-F | 9 | CACTTGGTCCTGCGCTTGA |
| h-Ubiquitin-R | 10 | CAATTGGGAATGCAACAACTTTAT |
| h-HSPA5-F | 11 | GACGGGCAAAGATGTCAGGA |
| h-HSPA5-R | 12 | GCCCGTTTGGCCTTTTCTAC |
| h-DNAJB9-F | 13 | TCTTAGGTGTGCCAAAATCGG |
| h-DNAJB9-R | 14 | TGTCAGGGTGGTACTTCATGG |
| h-sXBP1-F | 15 | TGCTGAGTCCGCAGCAGGTG |
| h-sXBP1-R | 16 | GCTGGCAGGCTCTGGGGAAG |
| h-XBP1-F | 17 | CCCTCCAGAACATCTCCCCAT |
| h-XBP1-R | 18 | ACATGACTGGGTCCAAGTTGT |
| h-PHGDH-F | 19 | CACGACAGGCTTGCTGAATGA |
| h-PHGDH-R | 20 | CTTCCGTAAACACGTCCAGTG |
| h-PSAT1-F | 21 | ACAGGAGCTTGGTCAGCTAAG |
| h-PSAT1-R | 22 | CATGCACCGTCTCATTTGCG |
| h-PSPH-F | 23 | GAGGACGCGGTGTCAGAAAT |
| h-PSPH-R | 24 | GGTTGCTCTGCTATGAGTCTCT |
| h-IL-6 F | 25 | TACCCCCAGGAGAAGATTCC |
| h-IL-6-R | 26 | GCCATCTTTGGAAGGTTCAG |
| h-IFNA-F | 27 | CCCAGGAGGAGTTTGGCAA |
| h-IFNA-R | 28 | TGCTGGATCATCTCATGGAGG |
| h-CXCL10-F | 29 | AAGCAGTTAGCAAGGAAAGGTC |
| h-CXCL10-R | 30 | GACATATACTCCATGTAGGGAAGTGA |

### RNA-Sequencing Analysis

Total RNA was extracted from cells using the Qiagen RNeasy Plus Mini Kit. All samples were examined for RNA quality by Agilent Bioanalyzer 2100. Illumina libraries were constructed prepared using NEB low input library preparation kit. Multiplexed libraries generated and were pooled at equimolar concentration and single-end reads were sequenced on an Illumina HiSeq 2500 in the Weill Cornell Epigenomics Core Facility at the depth of 21-37 million fragments per sample. Sequencing quality was measured with fastp (44). Reads were then mapped reads in genes counted against the human genome (hg38) with STAR aligner and Gencode v21. Differential gene expression analysis was performed in R (45) using the edgeR package (46, 47). Genes with low expression levels (<3 cpm) were filtered from all downstream analyses. The Benjamini-Hochberg false discovery rate procedure was used to calculate the FDR. Genes with FDR<0.05 and log 2 (fold-change)>1 were considered significant. Volcano plot and Heatmap were generated by complex heatmap packages. Pathways analysis for differential regulated genes were performed in R using fgsea package and normalized gene enrichment score were used for plotting.

### Chemokine and Cytokine Measurement

Secreted chemokines and cytokines such as IFN-α (Mabtech: 3425-1H-20), IL-6 (Mabtech: 3460-1H-20) and CXCL4 (R&D: DY795) were quantified in the supernatant of pDC cultures using enzyme-linked immunosorbent assay (ELISA).

### Metabolism Assay

Intracellular pyruvate were determined in pDCs by pyruvate detection kit (Cayman chemicals: 700470) as per the manufacture protocol. pDC cultured in RPMI medium were washed in 1 ml of PBS and centrifuged at 10000×g for 5 min at 4° C. Supernatants were removed, and cells were deproteinated in 0.5 ml of 0.5M MPA on ice for 5 min, followed by centrifugation at 10000×g for 5 min at 4° C. The deproteinated samples were neutralized with 25 μl of potassium carbonate, and then centrifugation at 10000×g for 5 min at 4° C. The supernatant was removed and deproteinated samples were used for pyruvate assay. For ATP determination, ATP determination kit (Sigma: A22066) was used in pDCs extracts as per the manufacture protocol.

Gene Editing in Human pDCs

Human pDCs isolated from PBMCs were electroporated by adding 2×105 cells in suspension onto 150 nM sgRNA-CAS9 ribonucleoprotein complexes using Neon™ transfection system (thermofisher: MPK5000). All materials for sgRNA-Cas9 complex generation were purchased from Integrated DNA Technologies and prepared as instructed (48). Eighty hours post-transfection, genetic ablation of target genes was assessed via quantitative RT-PCR. The 20-nucleotide CRISPR-RNA (crRNA) targeting human XBP1 (Homo sapiens chromosome 22, GRCh38.p12, NC_000022.11) is directed at the genomic sequence 5′-CGGTGCGTAGTCTGGAGCTACGG-3′ (SEQ ID NO: 31; the 3 additional nucleotides highlighted in bold represent the protospacer adjacent motif, or PAM). This target sequence corresponds to exon 1 of the human XBP1 transcript and was manually chosen by identifying a 20-base pair fragment immediately upstream of the highlighted PAM (49). The most likely on- and off-target effects of the manually selected CRISPR sequence were then analyzed using the Broad Institute's Genetic Perturbation Platform (50). To validate the genomic editing capacity of the crRNA, quantitative RT-PCR was performed on total RNA isolated from cells transfected with sgRNA-Cas9 complexes containing the XBP1 crRNA described above. The primers for evaluating deletion efficacy are listed in Table 3.

### Statistical Analysis

All statistical analyses were performed using GraphPad Prism 9 software. Significance for pairwise correlation analysis was calculated using the Spearman's correlation coefficient (r). Comparisons between two groups were assessed using unpaired or paired (for matched comparisons) two-tailed Student's t-test, or non-parametric Mann-Whitney U-test. Each dot indicates individual donors. Data are presented as mean±sem. P values of <0.05 were considered to be statistically significant.

### Example 1. ER Stress Activates the UPR and Inhibits IFN-α in pDCs Via the IRE1α-XBP Pathway

pDCs have an extensive endoplasmic reticulum (ER) (21) and produce copious amount of IFN-I in response to TLR7 and TLR9 signaling (3, 4). The effect of the UPR on pDC activity using two pharmacological inducers of ER stress: tunicamycin or thapsigargin (16, 22, 23) was tested. As shown in FIGS. 5A and 5B, a strong induction of the unfolded protein response (UPR), as measured by RNA-seq analysis was observed, with the induction of key genes associated with the UPR and ER stress pathways (FIGS. 5C and 5D), but their capacity to express IFN-α was not impacted in this setting (FIGS. 1A and 1B). This result is in sharp contrast with previous reports using mouse macrophages (20). Furthermore, both tunicamycin and thapsigargin drastically inhibited the expression levels of IFNA by pDCs in response to TLR9 (FIG. 1A), while negligible effects on viability (FIG. 5G) or IL-6 expression were detected (FIG. 5F). This was not due to a delay in the kinetic of IFN-α response as similar inhibition was observed at the protein level in an overnight assay (FIG. 1B). Similar inhibition by the UPR of the IFN-α secretion was observed when a TLR7 agonist was used (FIG. 5E). Transcriptomic analyses revealed that tunicamycin inhibited IFN-I genes and interferon-stimulated genes (ISGs) while inducing a strong UPR (FIGS. 1C-1E) and identified that the UPR activation was engaging the IRE1α arm (FIG. 1E).

Hence, disabling IRE1α using 2 independent inhibitors of its RNase domain, 4μ8c and MKC8866 (22, 23) (FIG. 6A and FIG. 6B), prevented ER stress-driven inhibition of IFN-α in TLR9-activated pDCs (FIGS. 1F and 1G and FIGS. 6C and 6D). Genetic loss of XBP1 in pDCs (FIG. 6E) which prevented optimal XBP1 splicing (FIG. 6F) also rescued IFNA expression by TLR9-stimulated pDCs facing ER stress (FIG. 6G), demonstrating that canonical IRE1-XBP1 signaling mediates this process. In contrast, inhibiting the other branches of the UPR, PERK or ATF6, had no effect on tunicamycin-induced XBP1 splicing (FIG. 6J) nor on the inhibition of IFN-α (FIGS. 611 and 6I). To further confirm the contribution by the IRE1α-XPB1 arm of the UPR to pDC responses, a gain of function approach was used. pDCs were incubated with IXA4, a small molecule that has been demonstrated to selectively activate IRE1α/XBP1 signaling without interfering with other arms of the UPR (24). As shown in FIG. 1H, IXA4 enhanced XBP1 splicing and simultaneously abrogated IFNA expression in TLR9-activated pDCs (FIG. 1I).

Taken together, these data indicate that the ER stress activates the UPR, which inhibits IFN-I response in activated pDCs through IRE1α-XBP1 signaling.

### Example 2. Fueling the TCA Cycle and ATP Generation are Required for TLR9-Induced IFN-I Response by pDCs

The spliced XBP1 isoform generated by IRE1α encodes the functional transcription factor XBP1, which induces factors implicated in restoring ER proteostasis while controlling diverse metabolic programs (25, 26). Using gene set enrichment analysis (GSEA), it was observed that transcriptional networks implicated in amino acid biosynthesis were markedly activated in pDCs experiencing ER stress, with or without TLR9 agonist treatment (FIGS. 2A and 2B). Further analysis showed that gene programs related to serine amino acid biosynthesis are highly enriched among all amino acid pathways (FIG. 2C and FIGS. 7A and 7B). The induction of some of these genes by both tunicamycin or thapsigargin was confirmed, irrespective of TLR9 signaling (FIG. 2D and FIGS. 7C and 7D). Of particular interest, tunicamycin or thapsigargin treatment markedly induced the gene encoding phosphoglycerate dehydrogenase (PHGDH) in pDCs (FIG. 2D). This enzyme transforms 3-phosphoglycerate into phosphohydroxypyruvate, which subsequently converts into serine (FIG. 2G) via transamination and phosphate ester hydrolysis reactions driven by PSAT1 and PSPH, respectively (27-29). Thus, a direct link was established between IRE1α-XBP1 and the induction of these metabolic regulated genes, as ER stress-driven induction of PHGDH, PSAT1, and PSPH was markedly inhibited upon abrogation of IRE1α-XBP1 signaling (FIG. 2E and FIGS. 7E-7G) and was conversely induced in pDCs treated with IXA4 (FIG. 2F). The impact of the IRE1α-XBP1-induced expression of PHGDH on pDCs activation was evaluated by inhibiting PHGDH enzymatic activity. Similarly to IRE1α inhibition (FIGS. 1F and 1G), abrogating PHGDH activity using NCT-503 (30) restored IFNA expression by TLR9-activated pDCs facing ER stress (FIGS. 2H and 2I). Since increased expression of PHGDH can boost serine biosynthesis, it may also cause a deficiency in pyruvate levels by shunting glycolysis (FIG. 2G) and the impact of both pathways on pDCs activity was evaluated. First, it was observed that IFN-α secretion by TLR9-activated pDCs was unaltered upon exogenous supplementation with L-serine (FIG. 2J), suggesting that elevated L-serine is not involved in ER stress-mediated IFN-α inhibition. In contrast, the intracellular pyruvate levels were significantly reduced in ER stressed-pDCs, and these levels could be restored by blocking PHGDH activity (FIGS. 2K and 2L). Further confirming these observations, exogenous pyruvate supplementation was sufficient to restore the IFNA expression by pDCs under ER stress condition (FIG. 3A and FIG. 3B). When produced by the cells, pyruvate enters the mitochondrion to fuel the tricarboxylic acid cycle (TCA) where it is converted to α-ketoglutarate (α-KG) and other TCA cycle substrates, to ultimately produce ATP by the electron transport chain (31-33) and this process has been shown to contribute to immune cell activation (34). It was observed that treatment with a cell-permeable analog of α-KG (35) also rescued the IFNA expression by TLR9-activated pDCs undergoing ER stress (FIGS. 3C and 3D). Consistent with these findings, it was observed that ER stress reduced intracellular ATP levels (FIGS. 3E-3H) and that supplementing either pyruvate (FIGS. 3E and 3F) or α-KG (FIGS. 3G and 3H) reversed that inhibition and restored the ATP levels. Of note, this was directly linked to increased expression of PHGDH during ER stress, as blocking its activity with NCT-503 similarly restored the intracellular ATP levels (FIG. 3I).

Using an inhibitor of both α-ketoglutarate dehydrogenase (KGDH) and pyruvate dehydrogenase (PDH), called CPI-613 (6,8-bis-benzylthio-octanoic acid), it was tested whether disrupting the TCA cycle could impact the IFN-α response by pDCs. CPI-613 has been well characterized and is in clinical trials for pancreatic cancer (36, 37). As shown in FIGS. 3J and 3K, CPI-613 inhibited the expression and secretion of IFN-α. As shown in FIGS. 8A-8E, CPI-613 also inhibited the expression of ISGs such as GBP1, IRF7, ISG54, MxB, CXCL10, while it had no effect on cell viability (FIG. 8F), or on the expression of PHGDH or on XBP1 splicing (FIGS. 8G and 8H). Consistent with this data, CPI-613 also reduced intracellular ATP levels in TLR9-activated pDCs (FIG. 3L).

Collectively, these data indicate that pyruvate and α-KG are key intermediate metabolites in the TCA cycle that are required for optimal IFN-α responses in TLR9-activated pDCs, and that this process is markedly blunted upon ER stress-driven activation of IRE1-XBP1 signaling due to the increased activity of PHGDH.

### Example 3. Dysregulated ER Stress in pDCs from Patients with SSc is Associated with Chronic IFN-I Response

Further experiments were done to elucidate the mechanisms underlying the chronic activation of pDCs in autoimmune patients. As shown in FIG. 4A, pDCs from patients with systemic sclerosis (SSc) were shown to have decreased expression of several UPR marker genes, including HSPA5, DNAJB9, XBP1, and XBP1s. Of note, there was an inverse correlation between the levels of these UPR genes and the expression of IFNA transcripts in pDCs from SSc patients (FIGS. 9A-9C). Furthermore, the expression level of PHGDH was significantly reduced in these cells as compared with pDCs from healthy donors (FIG. 4B) which is consistent with our observation linking ER stress and expression of this metabolic gene (FIGS. 2E and 2F). Strikingly, it was observed that CXCL4 could restore IFN-α expression and secretion by TLR9-activated by pDCs undergoing ER stress (FIG. 4C and FIG. 10A). Furthermore, CXCL4 exposure led to the significant decrease of the expression of UPR genes facing ER stress (FIG. 4D), and also dampened the PHGDH upregulation in this setting (FIG. 4E), which consistent with the normalization of the IFN-α response.

Patients with SSc have been shown to have elevated serum levels of CXCL4 (38, 39) which may explain the lower basal expression level of the UPR genes. Moreover, the induction of IFN-α by TLR9-stimulated pDCs in the presence of CXCL4 was abrogated upon treatment with CPI-613 (FIG. 4F), confirming the role of mitochondrial metabolism and the need for a functional metabolic response in this process. Finally, to further demonstrate that dysregulation of the TCA cycle is responsible for the hyperactivated status of pDCs of patients with SSc, patient cells were treated with CPI-613. It was observed that the expression of IFN-inducible genes by pDCs of SSc patients were drastically reduced by CPI-613 (FIG. 4G). Interestingly, CPI-613 had no effect on the secretion of CXCL4 itself (FIG. 4H), which suggests that either CXCL4 secretion is not dependent on similar metabolic control as the IFN-I response, or that its expression uses a different signaling cascade than TLR7 and 9. The latter hypothesis is consistent with an earlier report that TLR7 or TLR9 signaling does not induce CXCL4 in pDCs (13). Without being bound by theory, it is believed that as the systemic levels of CXCL4 is higher in patients with SSc (38, 39), the source of CXCL4 is not restricted to pDCs, and the observations above of a reduced UPR and of PHGDH in pDCs of patients, may not be due to an autocrine effect.

These data indicate that CXCL4 operates as a negative regulator of the UPR in pDCs, and that the elevated systemic levels of CXCL4 observed in patients leads to improper UPR, thereby promoting the hyperactivation of pDCs in patients with SSc. Although the mode of action and cellular sources of CXCL4 responsible for this effect are still unclear, restoring IRE1α-XBP1 signaling or inhibiting mitochondrial metabolism in pDCs may be useful approaches to interrupt the chronic activation status of these cells. Taken together, these data thus support the rationale for the use of IRE1α-XBP1 signaling activators (e.g., tunicamycin and thapsigargin), or TCA cycle disruptors (e.g., CPI-613) as a novel strategy for the treatment of patients with SSc or other autoimmune diseases.

### Example 4. TCA Inhibitors UK5099 and CB839 (Telagenastat) Inhibit Production of IFNA

As shown in Examples 2-3, CPI-613, a TCA inhibitor reduces IFNA expression. The ability of other TCA inhibitors, i.e., a pyruvate transporter and an inhibitor of glutaminase were also tested. Inhibitors of the TCA cycle are shown in FIG. 12. Purified pDCs from Healthy Donors (HDs) were first cultured with medium only or with an inhibitor for pyruvate transporter (UK5099 at 10, 20, and 40 μM) or with the inhibitor of glutaminase (CB839 at 0.5 μM) for 1 h when the TLR9 agonist (CpG-C274 at 0.075 μM) was added to the culture. FIG. 11A shows the cell viability at 6 h by flow cytometry. FIGS. 11B-11C show gene expression levels of IFNA as quantified at 5 h and normalized to TLR9 agonist treatment.

Cell viability using Flow cytometry. After 6 h of cell culture, pDCs were washed in PBS and then resuspended in FACS buffer and stained with DAPI. Cells were acquired by a fluorescence activated cell sorter (FACS) and analysis was performed using FlowJo analysis software. The gating strategy for viable cells involved progressively measuring total cells without uptake of DAPI.

RNA extraction and RT-PCR. After 6 h of cell culture, pDCs were lysed for total RNA extraction using the Qiagen RNeasy Plus Mini Kit. Quantity of RNA was measured by Nanodrop, and high-capacity cDNA Reverse Transcription kit (Thermofisher) was used to generate cDNA. qPCR reactions were performed. Gene expression levels were calculated based on relative threshold cycle (Ct) values as described. This was done using the formula Relative Ct=100 or 1000×1.8 (HSK-GENE), where HSK is the mean CT of duplicate housekeeping gene runs (Ubiquitin), GENE is the mean CT of duplicate runs of the gene of interest, and 100/1000 is arbitrarily chosen as a factor to bring all values above 0. Primers are shown in Table 3.

These results indicate that TCA cycle inhibitors can reduce the expression of IFN-I in TLR9-activated pDCs (FIGS. 11B-C) without affecting the viability of the cells (FIG. 11A). Further, there is an additive effect on the reduction of expression of IFN-I in TLR9-activated pDCs when both pyruvate transporter and glutaminase are blocked together (FIG. 11B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Y J. Liu, IPC: professional type 1 interferon-producing cells and plasmacytoid dendritic cell precursors. Annu Rev Immunol 23, 275-306 (2005).
2. M. Swiecki, M. Colonna, The multifaceted biology of plasmacytoid dendritic cells. Nat Rev Immunol 15, 471-485 (2015).
3. B. Reizis, Plasmacytoid Dendritic Cells: Development, Regulation, and Function. Immunity 50, 37-50 (2019).
4. F. J. Barrat, L. Su, A pathogenic role of plasmacytoid dendritic cells in autoimmunity and chronic viral infection. J Exp Med 216, 1974-1985 (2019).
5. M. Swiecki, S. Gilfillan, W. Vermi, Y Wang, M. Colonna, Plasmacytoid dendritic cell ablation impacts early interferon responses and antiviral NK and CD8(+) T cell accrual. Immunity 33, 955-966 (2010).
6. L. Cervantes-Barragan et al., Plasmacytoid dendritic cells control T-cell response to chronic viral infection. Proc Natl Acad Sci USA 109, 3012-3017 (2012).
7. F. O. Nestle et al., Plasmacytoid predendritic cells initiate psoriasis through interferon-alpha production. J Exp Med 202, 135-143 (2005).
8. J. Wenzel, T. Tuting, An IFN-Associated Cytotoxic Cellular Immune Response against Viral, Self-, or Tumor Antigens Is a Common Pathogenetic Feature in “Interface Dermatitis”. J Invest Dermatol 128, 2392-2402 (2008).
9. C. Guiducci et al., TLR recognition of self nucleic acids hampers glucocorticoid activity in lupus. Nature 465, 937-941 (2010).
10. V. Sisirak et al., Genetic evidence for the role of plasmacytoid dendritic cells in systemic lupus erythematosus. J Exp Med 211, 1969-1976 (2014).
11. S. L. Rowland et al., Early, transient depletion of plasmacytoid dendritic cells ameliorates autoimmunity in a lupus model. J Exp Med 211, 1977-1991 (2014).
12. C. Conrad et al., TNF blockade induces a dysregulated type I interferon response without autoimmunity in paradoxical psoriasis. Nature communications 9, 25 (2018).
13. M. D. Ah Kioon et al., Plasmacytoid dendritic cells promote systemic sclerosis with a key role for TLR8. Sci Transl Med 10, (2018).
14. R. Furie et al., Monoclonal antibody targeting BDCA2 ameliorates skin lesions in systemic lupus erythematosus. J Clin Invest 129, 1359-1371 (2019).
15. C. Hetz, K. Zhang, R. J. Kaufman, Mechanisms, regulation and functions of the unfolded protein response. Nat Rev Mol Cell Biol 21, 421-438 (2020).
16. S. E. Bettigole, L. H. Glimcher, Endoplasmic reticulum stress in immunity.

Annu Rev Immunol 33, 107-138 (2015).
17. J. R. Cubillos-Ruiz et al., ER Stress Sensor XBP1 Controls Anti-tumor Immunity by Disrupting Dendritic Cell Homeostasis. Cell 161, 1527-1538 (2015).
18. A. M. Reimold et al., Plasma cell differentiation requires the transcription factor XBP-1. Nature 412, 300-307 (2001).
19. N. N. Iwakoshi, M. Pypaert, L. H. Glimcher, The transcription factor XBP-1 is essential for the development and survival of dendritic cells. J Exp Med 204, 2267-2275 (2007).
20. J. Moretti et al., STING Senses Microbial Viability to Orchestrate Stress-Mediated Autophagy of the Endoplasmic Reticulum. Cell 171, 809-823 e813 (2017).
21. F. P. Siegal et al., The nature of the principal type 1 interferon-producing cells in human blood. Science 284, 1835-1837 (1999).
22. M. Song et al., IRE1alpha-XBP1 controls T cell function in ovarian cancer by regulating mitochondrial activity. Nature 562, 423-428 (2018).
23. S. Chopra et al., IRE1alpha-XBP1 signaling in leukocytes controls prostaglandin biosynthesis and pain. Science 365, (2019).
24. J. M. D. Grandjean et al., Pharmacologic IRE1/XBP1s activation confers targeted ER proteostasis reprogramming. Nature chemical biology 16, 1052-1061 (2020).
25. H. C. Liou et al., A new member of the leucine zipper class of proteins that binds to the HLA DR alpha promoter. Science 247, 1581-1584 (1990).
26. L. H. Glimcher, XBP1: the last two decades. Ann Rheum Dis 69 Suppl 1, i67-71(2010).
27. R. Possemato et al., Functional genomics reveal that the serine synthesis pathway is essential in breast cancer. Nature 476, 346-350 (2011).
28. J. W. Locasale et al., Phosphoglycerate dehydrogenase diverts glycolytic flux and contributes to oncogenesis. Nat Genet 43, 869-874 (2011).
29. Q. Spillier, R. Frederick, Phosphoglycerate dehydrogenase (PHGDH) inhibitors: a comprehensive review 2015-2020. Expert Opin Ther Pat, 1-12 (2021).
30. M. E. Pacold et al., A PHGDH inhibitor reveals coordination of serine synthesis and one-carbon unit fate. Nature chemical biology 12, 452-458 (2016).
31. E. J. Pearce, B. Everts, Dendritic cell metabolism. Nat Rev Immunol 15, 18-29 (2015).
32. D. Wu et al., Type 1 Interferons Induce Changes in Core Metabolism that Are Critical for Immune Function. Immunity 44, 1325-1336 (2016).
33. I. Martinez-Reyes, N. S. Chandel, Mitochondrial TCA cycle metabolites control physiology and disease. Nature communications 11, 102 (2020).
34. B. Everts et al., TLR-driven early glycolytic reprogramming via the kinases TBK1-IKKvarepsilon supports the anabolic demands of dendritic cell activation. Nat Immunol 15, 323-332 (2014).
35. J. M. Kee, R. C. Oslund, D. H. Perlman, T. W. Muir, Apan-specific antibody for direct detection of protein histidine phosphorylation. Nature chemical biology 9, 416-421 (2013).
36. S. D. Stuart et al., A strategically designed small molecule attacks alpha-ketoglutarate dehydrogenase in tumor cells through a redox process. Cancer Metab 2, 4 (2014).
37. P. A. Philip et al., A Phase III open-label trial to evaluate efficacy and safety of CPI-613 plus modified FOLFIRINOX (mFFX) versus FOLFIRINOX (FFX) in patients with metastatic adenocarcinoma of the pancreas. Future Oncol 15, 3189-3196 (2019).
38. L. van Bon et al., Proteome-wide analysis and CXCL4 as a biomarker in systemic sclerosis. N Engl J Med 370, 433-443 (2014).
39. E. R. Volkmann et al., Changes in plasma CXCL4 levels are associated with improvements in lung function in patients receiving immunosuppressive therapy for systemic sclerosis-related interstitial lung disease. Arthritis Res Ther 18, 305 (2016).
40. F. van den Hoogen et al., 2013 classification criteria for systemic sclerosis: an American College of Rheumatology/European League against Rheumatism collaborative initiative. Arthritis Rheum 65, 2737-2747 (2013).
41. E. C. LeRoy, T. A. Medsger, Jr., Criteria for the classification of early systemic sclerosis. J Rheumatol 28, 1573-1576 (2001).
42. C. Guiducci et al., Properties regulating the nature of the plasmacytoid dendritic cell response to Toll-like receptor 9 activation. J Exp Med 203, 1999-2008 (2006).
43. F. J. Barrat et al., Nucleic Acids of Mammalian Origin Can Act as Endogenous Ligands for Toll-like Receptors and May Promote Systemic Lupus Erythematosus. J Exp Med 202, 1131-1139 (2005).
44. S. Chen, Y Zhou, Y Chen, J. Gu, fastp: an ultra-fast all-in-one FASTQ preprocessor. Bioinformatics 34, i884-i890 (2018).
45. R. C. Team, R: A language and environment for statistical computing. R Foundation for Statistical Computing, Vienna, Austria. http://www.r-project.org/, (2020).
46. M. D. Robinson, D. J. McCarthy, G. K. Smyth, edgeR: a Bioconductor package for differential expression analysis of digital gene expression data.

Bioinformatics 26, 139-140 (2010).
47. Z. Gu, R. Eils, M. Schlesner, Complex heatmaps reveal patterns and correlations in multidimensional genomic data. Bioinformatics 32, 2847-2849 (2016).
48. https://sfvideo.blob.core.windows.net/sitefinity/docs/default-source/protocol/alt-r-crispr-cas9-user-guide-ribonucleoprotein-electroporation-amaxa-nucleofector-system6a01611532796e2eaa53ff00001c1b3c.pdf?sfvrsn=71c43407_26.
49. F. A. Ran et al., Genome engineering using the CRISPR-Cas9 system. Nat Protoc 8, 2281-2308 (2013).
50. https://portals.broadinstitute.org/gpp/public/analysis-tools/%20sgrna-design.

1. A method of treating an autoimmune disease in a human subject in need thereof, the method comprising administering to the subject a therapeutically effective amount of a compound that activates the Unfolded Protein response (UPR) in immune cells in the subject.

2. A method of treating an autoimmune disease in a human subject in need thereof, the method comprising administering to the subject a therapeutically effective amount of a compound that disrupts the tri-carboxylic acid (TCA) cycle in immune cells.

3-4. (canceled)

5. The method of claim 1, wherein the compound activates the IRE1α-XBP1 signaling branch of the UPR in immune cells.

6. The method of claim 1, wherein the immune cells are dendritic cells, macrophages, T cells, B cells, natural killer cells, and/or neutrophils.

7. The method of claim 1, wherein the compound that activates the UPR is tunicamycin, thapsigargin, or IXA4.

8. The method of claim 2, wherein the compound that disrupts the tri-carboxylic acid (TCA) cycle is
(a) a compound of Formula I 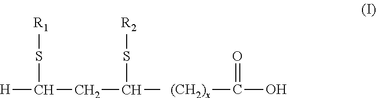
or a pharmaceutically acceptable salt thereof,
wherein R₁ and R₂ are independently selected from the group consisting of acyl defined as R₃C(0)-, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10,
(b) UK5099 (PF-1005023) 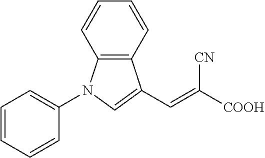 [SMILES: N#C/C(=C\c1cn(-c2ccccc2)c2ccccc12)C(=O)O]
or (c) CB839 (Telagenastat)

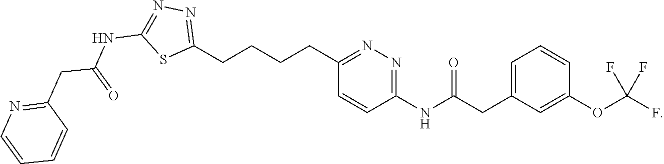 [SMILES: O=C(Cc1cccc(OC(F)(F)F)c1)Nc1ccc(CCCCc2nnc(NC(=O)Cc3ccccn3)s2)nn1]

9. The method of claim 8, wherein R₁ and R₂ are benzyl or benzoyl.

10. The method of claim 7, wherein the compound of Formula I is

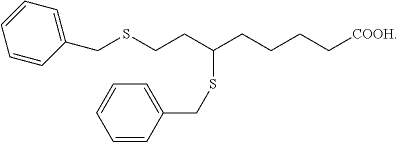 [SMILES: O=C(O)CCCCC(CCSCc1ccccc1)SCc1ccccc1]

11. The method of claim 8, wherein the compound of formula I is 6,8-bis-benzylthio-octanoic acid.

12. The method of claim 1, wherein the autoimmune disease is systemic sclerosis (scleroderma), systemic lupus erythematosus (SLE), rheumatoid arthritis (RA), Sjogren's syndrome, discoid lupus, cutaneous lupus, lupus nephritis, inflammatory bowel disease, psoriasis, type I diabetes, dermatomyositis, or polymyositis.

13. The method of claim 1, wherein the subject is concurrently treated with one or more agents selected from the group consisting of a nonsteroidal anti-inflammatory drug (NSAID), an immunosuppressant, a corticosteroid, an antimalarial, a fusion protein, and an antibody.

14. The method of claim 13, wherein the immunosuppressant is methotrexate, mycophenolate mofetil (MIMF), cyclophosphamide, cyclosporin, or azathioprine.

15-18. (canceled)

19. The method of claim 1, wherein the treatment reduces production of inflammatory cytokines or chemokines by dendritic cells in the human subject.

20. The method of claim 19, wherein the inflammatory cytokines or chemokines are selected from the group consisting of: type I interferon (IFN-I), IL-6, or TNF-α, type III interferon, MIP-1a/CCL3, MIP-1/CCL4, CCL5/RANTES, and IP-10/CXCL10.

21. The method of claim 6, wherein the dendritic cells are plasmocytoid dendritic cells.

22. The method of claim 21, wherein the dendritic cells express one or more of CD123, CD303 (BDCA2), CD304 (BDCA4), and immunoglobulin-like transcript 7 (ILT7).

23. The method of claim 21, wherein the dendritic cells do not express the lineage-associated markers (Lin) CD3, CD19, CD14, CD16 and CD11c.

24. The method of claim 20, wherein the method inhibits and/or reduces IFN-I production in the human subject in need thereof by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the human subject or in a control.

25. The method of claim 1, wherein the treatment reduces the expression of one or more of the interferon stimulated genes selected from the group consisting of Guanylate Binding Protein 1 (GBP1), Interferon Regulatory Factor 7 (IRF7), interferon stimulated gene 54 (ISG54), myxovirus resistance protein B (MxB), and 2′-5′-Oligoadenylate Synthetase 2 (OAS2).

26. The method of claim 1, wherein the treatment enhances expression of phosphoglycerate dehydrogenase (PHGDH), phosphoserine Phosphatase (PSPH), and phosphoserine Aminotransferase 1 (PSAT1).

27-29. (canceled)
